(19) United States
(12) Patent Application Publication
(10) Pub. No.: US 2024/0382467 A1
(43) Pub. Date: Nov. 21, 2024

(54) COMPOSITIONS AND METHODS FOR TREATING AND/OR PREVENTING LUNG INJURY

(71) Applicants: University of Virginia Patent Foundation, Charlottesville, VA (US); Old Dominion University, Norfolk, VA (US); OLD DOMINION UNIVERSITY RESEARCH FOUNDATION, Norfolk, VA (US)

(72) Inventors: John S. Lazo, Charlottesville, VA (US); Elizabeth R. Sharlow, Butler, PA (US); John D. Catravas, Virginia Beach, VA (US); Ruben ML Colunga Biancatelli, Norfolk, VA (US); Pavel A. Solopov, Virginia Beach, VA (US)

(73) Assignees: University of Virginia Patent Foundation, Charlottesville, VA (US); Old Dominion University, Norfolk, VA (US)

(21) Appl. No.: 18/286,976

(22) PCT Filed: Apr. 14, 2022
(86) PCT No.: PCT/US2022/024927

§ 371 Date: Oct. 13, 2023

Related U.S. Application Data
(60) Provisional application No. 63/174,750, filed on Apr. 14, 2021.

(51) Int. Cl. A61K 31/4365; A61P 31/14; A61P 39/00

(52) U.S. Cl. CPC A61K 31/4365; A61P 31/14; A61P 39/00

(57) ABSTRACT

Provided are methods for treating and/or preventing diseases, disorders, and/or conditions associated with viral infections in subjects, which in some embodiments can include administering to the subject an effective amount of a PTP4A3 inhibitor. In some embodiments, the disease, disorder, and/or condition is characterized by lung damage, ALI, ARDS, or any combination thereof. Also provided are methods for reducing or inhibiting virus-induced alveolar inflammation and/or damage, methods for reducing or inhibiting induction of inflammatory cytokines and/or chemokines in subjects, methods for reducing or inhibiting pulmonary diseases, disorders, and/or conditions associated with viral infections, or pulmonary damage resulting therefrom, methods for preventing and/or treating chemical damage to lungs, uses of PTP4A3 inhibitors in the presently disclosed methods.

### CROSS REFERENCE TO RELATED APPLICATION

The presently disclosed subject matter claims the benefit of U.S. Provisional Patent Application Ser. No. 63/174,750, filed Apr. 14, 2021, the disclosure of which incorporated herein by reference in its entirety.

### GOVERNMENT INTEREST

This invention was made with government support under Grant Nos. ES030674 and ES030528 awarded by the National Institutes of Health. The government has certain rights in the invention.

### REFERENCE TO SEQUENCE LISTING SUBMITTED ELECTRONICALLY

The content of the electronically submitted sequence listing in ASCII text file (Name: 3062_153_PCT_ST25.txt; Size: 15 kilobytes; and Date of Creation: Apr. 14, 2022) filed with the instant application is incorporated herein by reference in its entirety.

### BACKGROUND

Severe Acute Respiratory Syndrome Coronavirus-2 (SARS-CoV-2) is a Beta Coronaviridae responsible of the SARS-CoV-2-related disease (COVID-19) pandemic. Since its first appearance in Wuhan, China (Winkler et al., 2020; Zhou et al., 2020), it has infected more than 100,000,000 and provoked the death of over 2.2 million worldwide (COVID-19 Map—Johns Hopkins Coronavirus Resource Center). While vaccination programs have started, significant delays are observed resulting from limited stocks, problems with worldwide distribution and limited compliance by the populace. In addition, the emergence of new strains of SARS-CoV-2 could delay the achievement of herd immunity and further reduce the impact of current vaccines. It is thus prudent to continue investigating new therapeutic approaches to COVID-19.

The study of SARS-CoV-2 pathogenicity is challenging due to difficulties in the design of an appropriate animal model. ALI is an event after SARS-CoV-2 infection linked to the development of cytokine production and lethality, although the mediators of ALI are not well established. Just as with SARS-CoV-1, SARS-CoV-2 requires binding of its Spike protein to the host ectoenzyme, angiotensin converting enzyme (ACE2) for cellular entry (Michaud et al., 2020). While hamsters and monkeys express ACE2 (Munster et al., 2020; Sia et al., 2020), wild type mice, which are more commonly employed in basic research, do not (Oladunni et al., 2020). Transgenic mice expressing the human ACE2 gene under the control of the cytokeratin 18 promoter (K18-hACE2) have been created and they faithfully reproduce the pathology seen in humans when infected with the live SARS-CoV-2 (Bao et al., 2020; Oladunni et al., 2020; Winkler et al., 2020). Unfortunately, studies with live SARS-CoV-2 require Biosafety Level 3 (BSL-3) facilities for personnel protection thus making them more cumbersome and available to a limited group of investigators. The K18-hACE2 transgenic mice do, however, enable an investigation into the early effects of elements of the viral particle.

### SUMMARY

Ibis Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter relates to methods for treating and/or preventing diseases, disorders, and/or conditions associated with viral infections in subjects. In some embodiments, the methods comprise, consist essentially of, or consist of administering to a subject in need thereof an effective amount of a protein tyrosine phosphatase 4A3 (PTP4A3) inhibitor. In some embodiments, the disease, disorder, and/or condition comprises lung damage. In some embodiments, the disease, disorder, and/or condition associated with viral infection is selected from the group consisting of acute lung injury (ALI), acute respiratory distress syndrome (ARDS). In some embodiments, the PTP4A3 inhibitor is 7-imino-2-phenylthieno[3,2-c]pyridine-4,6(5H,7H)-dione, thienopyridone, pharmaceutically acceptable salts thereof, prodrugs thereof, metabolites thereof, or any combination thereof. In some embodiments, the PTP4A3 inhibitor has the following general structure:

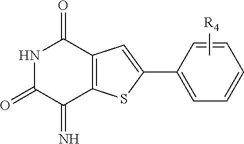

wherein:
R⁴ is selected from the group consisting of H. —OH, —OC₁₋₄ alkyl, —OR^{a}, trifluoroC₁₋₄alkoxy, —SC₁₋₄ alkyl. —SR^{a}, —SO₂—C₁₋₄ alkyl, —SO₂R^{a}, —SOC₁₋₄alkyl, —SOR^{a}, —SO₂NHR_{b}, —NR^{C}R^{d}, halo, —C₁₋₁₂alkyl, —C₂₋₆alkenyl, —C₂₋₆alkynyl, —C₃-6cycloalkyl, phenyl, benzyl, monocyclic heteroaryl optionally substituted with R^{b}, —C₃₋₆ cycloalkyl. —C₄₋₂heterocycloalkyl containing one or two of O, S, and N, —OC(O)R^{b}, —OC(O)R^{b}, —P(O)(OR^{b})₁-2, —P(S)(OR^{b})₁₋₂, —P(O)(NR^{C}R^{d})₁₋₂, —P(S)(NR^{C}R^{d})₁₋₂, —O(CH₂—CH₂—O)₁₋₄CH₃, —CN. —NO₂, —C(O)C₁₋₄alkyl, and —C(O)—R^{b},
R^{a} is selected from the group consisting of —C₃₋₆cycloalkyl. —C₂₋₆ alkenyl. —C₂-6 alkynyl, phenyl, benzyl, and monocyclic heteroaryl, wherein the phenyl, benzyl, or monocyclic heteroaryl is optionally substituted with R^{b};
R^{b} in each instance is independently selected from the group consisting of H, halo, —OH, —COOH, —C₁₋₁₂alkyl, phenyl, benzyl, and monocyclic heteroaryl, wherein the phenyl, benzyl, or monocyclic heteroaryl is optionally substituted with —C₂₋₆alkenyl. —C₂₋₆ alkynyl, trifluroC₁₋₄ alkoxy, —OC₁₋₄ alkyl, —O(CH₂—CH₂—O)₁₋₄ CH₃, —O-phenyl, —O— benzyl, —NC₁₋₄ alkyl, —N-phenyl, and —N-benzyl, or —N-monocyclic heteroaryl;
R^{C} and R^{d} are each independently selected from the group consisting of H. —C₁₋₄ alkyl. —C(O)—C₁₋₄alkyl, —C(O)—R^{e}, —C₁₋₄ alkyl-R^{a}, —SO₂—R^{a}, —SO₂—C₁₋₄ alkyl, phenyl, benzyl, and monocyclic heteroaryl, wherein the phenyl, benzyl, or monocyclic heteroaryl is optionally substituted with R^{b}, or
or R^{C} and R^{d} taken together with the nitrogen to which they are attached represent an optionally substituted monocyclic heterocycloalkyl containing one or more of O, S, and N.
   and/or is a pharmaceutically acceptable salt thereof, prodrug thereof, metabolite thereof, or any combination thereof.

In some embodiments, the viral infection is a SARS-CoV-2 infection and/or an influenza A infection, optionally an infection with an H1N1 subtype of influenza A.

In some embodiments, the presently disclosed subject matter also relates to methods for reducing or inhibiting virus-induced alveolar inflammation and/or damage. In some embodiments, the methods comprise, consist essentially of, or consist of administering to a subject in need thereof an effective amount of a PTP4A3 inhibitor. In some embodiments, the disease, disorder, and/or condition is induced by infection with SARS-CoV-2. In some embodiments, the disease, disorder, and/or condition is induced by infection with an influenza A virus, which in some embodiments can be an H1N1 subtype of influenza A. In some embodiments, the PTP4A3 inhibitor is 7-imino-2-phenylthieno[3,2-c]pyridine-4,6(5H,7H)-dione, thienopyridone, pharmaceutically acceptable salts thereof, prodrugs thereof, metabolites thereof, or any combination thereof. In some embodiments, the PTP4A3 inhibitor has the following general structure:

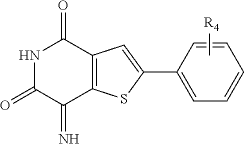

wherein:
R⁴ is selected from the group consisting of H, —OH. —OC₁₋₄ alkyl, —OR^{a}, trifluoroC₁₋₄alkoxy, —SC₁₋₄ alkyl, —SR^{a}, —SO₂—C₁-_alkyl. —SO₂R^{a}, —SOC₁₋₄ alkyl, —SOR^{a}, —SO₂NHR^{b}, —NR^{C}R^{d}, halo. —C₁₋₁₂ alkyl, —C₂₋₆ alkenyl. —C₂₋₆ alkynyl, —C₃₋₆cycloalkyl, phenyl, benzyl, monocyclic heteroaryl optionally substituted with R^{b}, —C₃₋₆ cycloalkyl, —C₄₋₇ heterocycloalkyl containing one or two of O, S, and N, —OC(O)R^{b}, —OC(O)R^{b}, —P(O)(OR^{b})₁₋₂, —P(S)OR^{b})₁₋₂, —P(O)(NR^{C}R^{d})₁₋₂, —P(S)(NR^{C}R^{d})₁₋₂, —O(CH₂—CH₂—O)₁₋₄CH₃, —CN, —NO₂, —C(O)C₁₋₄alkyl, and —C(O)—R^{b},
R^{a} is selected from the group consisting of —C₃₋₆cycloalkyl, —C₂₋₆alkenyl, —C₂₋₆, alkynyl, phenyl, benzyl, and monocyclic heteroaryl, wherein the phenyl, benzyl, or monocyclic heteroaryl is optionally substituted with R^{b};
R^{b} in each instance is independently selected from the group consisting of H, halo. —OH, —COOH, —C₁₋₁₂alkyl, phenyl, benzyl, and monocyclic heteroaryl, wherein the phenyl, benzyl, or monocyclic heteroaryl is optionally substituted with —C₂₋₆ alkenyl, —C₂₋₄ alkynyl, trifluroC₁₋₄alkoxy,-OC₁₋₄alkyl, —O(CH₂—CH₂—O)₁₋₄CH₃, —O-phenyl, —O— benzyl, —NC₁₋₄alkyl, —N-phenyl, and —N-benzyl, or —N-monocyclic heteroaryl:
R^{C} and R^{d} are each independently selected from the group consisting of H, —C₁₋₄alkyl, —C(O)—C₁₋₄alkyl, —C(O)—R^{e}, —C₁₋₄alkyl-R^{e}, —SO₂—R^{a}, —SO₂—C₁₋₄ alkyl, phenyl, benzyl, and monocyclic heteroaryl, wherein the phenyl, benzyl, or monocyclic heteroaryl is optionally substituted with R^{b}, or
or R^{C} and R^{d} taken together with the nitrogen to which they are attached represent an optionally substituted monocyclic heterocycloalkyl containing one or more of O, S, and N.
   and/or is a pharmaceutically acceptable salt thereof, prodrug thereof, metabolite thereof, or my combination thereof.

In same embodiments, the presently disclosed subject matter also relates to methods for reducing or inhibiting induction of inflammatory cytokines and/or chemokines by viral to infections. In some embodiments, the presently disclosed methods comprise, consist essentially of, or consist of administering to a subject in need thereof an effective amount of a PTP4A3 inhibitor. In some embodiments, the inflammatory cytokine and/or chemokine is selected from the group consisting of IFNγ, IL-1β, IL-6, IL-17, MCP-1, KC, TNFα, MIP-1α, CLL2, CCL3, and CXCL1. In some embodiments, the PTP4A3 inhibitor is 7-imino-2-phenylthieno[3,2-c]pyridine-4,6(5H,7H)-dione, thienopyridone, pharmaceutically acceptable salts thereof, prodrugs thereof, metabolites thereof, or any combination thereof. In some embodiments, the PTP4A3 inhibitor has the following general structure:

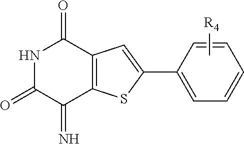

wherein:
R⁴ is selected from the group consisting of H. —OH, —OC₁₋₄ alkyl. —OR^{a}, trifluoroC₁₋₄alkoxy, —SC₁₋₄alkyl. —SR^{a}, —SO₂—C₁₋₄ alkyl, —SO₂R^{a}, —SOC₁₋₄ alkyl, —SOR^{a}, —SO₂NHR^{b}, —NR^{C}R^{d}, halo, —C₁₋₁₂ alkyl, —C₂₋₆alkenyl, —C₂₋₆alkynyI, —C₃₋₆ cycloalkyl, phenyl, benngi, monocyclic heteroaryl optionally substituted with R^{b}, —C₃₋₆cycloalkyl. —C₄₋₇ heterocycloalkyl containing one or two of O, S, and N, —OC(O)R^{b}, —OC(O)R^{b}, —P(O)(OR^{b})₁₋₂, —P(S)(OR^{b})₁₋₂, —P(O)(NR^{C}R^{d})₁₋₂, —P(S)(NR^{C}R^{d})₁₋₂, —O(CH₂—CH₂—O)₁₋₄CH₃, —CN, —NO₂, —C(O)C₁₋₄alkyl, and —C(O)—R^{b},
R^{a} is selected from the group consisting of —C₃₋₆ cycloalkyl, —C₂₋₆alkenyl, —C₂₋₆ alkynyl, phenyl, benzyl, and monocyclic heteroaryl, wherein the phenyl, benzyl, or monocyclic heteroaryl is optionally substituted with R^{b};
R^{b} in each instance is independently selected from the group consisting of H, halo. —OH, —COOH, —C₁₋₁₂ alkyl, phenyl, benzyl, and monocyclic heteroaryl, wherein the phenyl, benzyl, or monocyclic heteroaryl is optionally substituted with —C₂₋₆alkenyl, —C₂₋₆alkenyl, trifluroC₁₋₄ alkoxy, —OC₁₋₄ alkyl, —O(CH₂—CH₂—O)₁₋₄CH₃, —O-phenyl, —O-benzyl, —NC₁₋₄alkyl, —N-phenyl, and —N-benzyl, or —N-monocyclic heteraryl:
R^{C} and R^{d} are each independently selected from the group consisting of H, —C₁₋₄alkyl, —C(O)—C₁₋₄alkyl. —C(O)—R^{b}, —C₁₋₄alkyl-R^{a}, —SO₂—R^{a}, —SO₂—C₁₋₄alkyl, phenyl, benzyl, and monocyclic heteroaryl, wherein the phenyl, benzyl, or monocyclic heteroryl is optionally substituted with R^{b}, or
or R^{C} and R^{d} taken together with the nitrogen to which they are attached represent an optionally substituted monocyclic heterocycloalkyl containing one or more of O, S, and N.
   and/or is a pharmaceutically acceptable salt thereof, prodrug thereof metabolite thereof, or may combination thereof.

In some embodiments, the presently disclosed subject matter also relates to methods for reducing or inhibiting a pulmonary disease, disorder, and/or condition associated with viral infections in a subject, or pulmonary damage resulting therefrom. In some embodiments, the methods comprise, consist essentially of, or consist of administering to a subject in need thereof an effective amount of a PTP4A3 inhibitor. In some embodiments, the pulmonary disease, disorder, and/or condition is alveolar thickening, neutrophil infiltration, endothelial barrier disruption, fibrosis, or may combination thereof. In some embodiments, the PTP4A3 inhibitor is 7-imino-2-phenylthieno[3,2-c]pyridine-4,6(5H,7H)-dione, thienopyridone, pharmaceutically acceptable salts thereof, prodrugs thereof, metabolites thereof, or may combination thereof. In some embodiments, the PTP4A3 inhibitor has the following general structure:

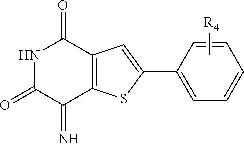

wberein:
R⁴ is selected from the group consisting of H, —OH. —OC₁₋₄ alkyl, —OR^{a}, trifluoroC₁₋₄alkoxy, —SC₁₋₄alkyl, —SR^{a}, —SO₂—C₁₋₄alkyl. —SO₂R^{a}, —SOC₁₋₄ alkyl, —SOR^{a}, —SO₂NHR^{b}, —NR^{C}R^{d}, halo. —C₁₋₁₂alkyl, —C₂₋₆alkenyl. —C₂₋₆ alkynyl, —C₃₋₆cycloalkyl, phenyl, benzyl, monocyclic heteroaryl optionally substituted with R^{b}, —C₃₋₆ cycloalkyl, —C₄₋₇ heterocycloalkyl containing one or two of O, S, and N, —OC(O)R^{b}, —OC(O)R^{b}, —P(O)(OR^{b})₁₋₂, —P(S)OR^{b})₁₋₂, —P(O)(NR^{C}R^{d})₁₋₂, —P(S)(NR^{C}R^{d})₁₋₂, —O(CH₂—CH₂—O)₁₋₄CH₃, —CN, —NO₂, —C(O)C₁₋₄alkyl, and —C(O)—R^{b},
R^{a} is selected from the group consisting of —C₃₋₆cycloalkyl, —C₂₋₆alkenyl, —C₂₋₆ alkynyl, phenyl, benzyl, and monocyclic heteroaryl. % herein the phenyl, benzyl, or monocyclic heteroaryl is optionally substituted with R^{b};
R^{b} in each instance is independently selected from the group consisting of H, halo. —OH, —COOH, —C₁₋₁₂alkyl, phenyl, benzyl, and monocyclic heteroaryl, wherein the phenyl, benzyl, or monocyclic heteroaryl is optionally substituted with —C₂₋₆ alkenyl, —C₂₋₆ alkynyl, trifluroC₁₋₄alkoxy, —OC₁₋₄alkyl. —O(CH₂—CH—O)₁₋₄CH, —O-phenyl, —O— benzyl, —NC₁₋₄alkyl, —N-phenyl, and —N-benzyl, or —N-monocyclic heteroaryl:
R^{C} and R^{d} are each independently selected from the group consisting of H, —C₁₋₄ alkyl, —C(O)—C₁₋₄alkyl. —C(O)—R^{e}—C₁₋₄alkyl-Rₑ, —SO₂—R^{a}, —SO₂—C₁₋₄ alkyl, phenyl, benzyl, and monocyclic heteroaryl, wherein the phenyl, benzyl, or monocyclic heteroaryl is optionally substituted with R^{b}, or
or R^{C} and R^{d} taken together with the nitrogen to which they are attached represent an optionally substituted monocyclic heterocycloalkyl containing one or more of O, S, and N.
   and/or is a pharmaceutically acceptable salt thereof, prodrug thereof metabolite thereof, or may combination thereof. In some embodiments, the viral infection is a SARS-CoV-2 infection and/or an influenza A infection, optionally an infection with an H1N1 subtype of influenza A. In some embodiments, the administering is oral, intravenous, intramuscular, subcutaneous intraperitoneal, intranasal, pulmonary, or ay combination thereof.

In some embodiments, the presently disclosed subject matter also relates to uses of PTP4A3 inhibitors for treating and/or preventing diseases, disorders, and/or conditions associated with viral infection in a subject, and/or pulmonary damage resulting therefrom. In some embodiments, the disease, disorder, and/or condition associated with the viral infection is selected from the group consisting of ALI and ARDS. In some embodiments the PTP4A3 inhibitor is 7-imino-2-phenylthieno[3,2-c]pyridine-4,6(5H,7H)-dione, thienopyridone, a pharmaceutically acceptable salt thereof a prodrug thereof, a metabolite thereof, or ay combination thereof. In some embodiments, the PTP4A3 inhibitor has the following general structure:

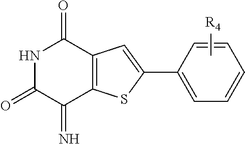

wherein:
R⁴ is selected from the group consisting of H. —OH, —OC₁₋₄ alkyl, —OR^{a}, trifluoroC₁₋₄ alkoxy, —SC₁₋₄ alkyl. —SR^{a}, —SO₂—C₁₋₄ alkyl, —SO₂R^{a}, —SOC₁₋₄ alkyl, —SOR^{a}, —SO₂NHR^{b}, NR^{C}R^{d}, halo, —C₁₋₁₂ alkyl, —C₂₋₆ alkenyl, —C₂₋₆alkynyl, —C₃₋₆ cycloalkyl, phenyl, benzyl, monocyclic heteroaryl optionally substituted with R^{b}, —C₃₋₆ cycloalkyl. —C₄₋₇ heterocycloalkyl containing one or two of O, S, and N, —OC(O)R^{b}, —OC(O)R^{b}, —P(O)(OR)₁₋₂, —P(S)(OR^{b})₁₋₂, —P(O)(NR^{C}R^{d})₁₋₂, —P(S)(NR^{C}R^{d})₁₋₂, —O(CH₂—CH₂—O)₁₋₄CH₃, —CN, —NO₂, —C(O)C₁₋₄alkyl, and —C(O)—R^{b},
R^{a} is selected from the group consisting of —C₃₋₆cycloalkyl, —C₂₋₆alkenyl, —C₂₋₆ alkynyl, phenyl, benzyl, and monocyclic heteroaryl, wherein the phenyl, benzyl, or monocycic heteroaryl is optionally substituted with R^{b};
R^{b} in each instance is independently selected from the group consisting of H, halo, —OH, —COOH. —C₁₋₁₂alkyl, phenyl, benzyl, and monocyclic heteroaryl, wherein the phenyl, benzyl, or monocyclic heteroaryl is optionally substituted with —C₂₋₄ alkenyl. —C₁₋₄ alkynyl, trifluoC₁₋₄alkoxy, —OC₁₋₄ alkyl, —O(CH—CH₂—O)₁₋₄ CH₃, —O-phenyl, —O— benzyl, —NC₁₋₄ alkyl, —N-phenyl, and —N-benzyl, or —N-monocyclic heteroaryl;
R^{C} and R^{d} are each independently selected from the group consisting of H, —C₁₋₄alkyl, —C(O)—C₁₋₄alkyl, —C(O)—R^{e}, —C₁₋₄alkyl-R^{e}, SO₂—R^{a}, —SO₂—C₁₋₄alkyl, phenyl, benzyl, and monocyclic heteroaryl, wherein the phenyl, benzyl, or monocyclic heteroaryl is optionally substituted with R^{b}, or
or R^{C} and R^{d} taken together with the nitrogen to which they are attached represent an optionally substituted monocyclic heterocycloalkyl containing one or more of O, S, and N.
   and/or is a pharmaceutically acceptable salt thereof, prodrug thereof; metabolite thereof, or any combination thereof.

In some embodiments, the presently disclosed subject matter also relates to compositions comprising, consisting essentially of, or consisting of PTP4A3 inhibitors for use in methods for treating and/or preventing diseases, disorders, and/or conditions associated with viral infection in subjects, optionally a SARS-CoV-2 infection in the subject. In some embodiments, the methods comprise, consist essentially of, or consist of administering to a subject infected with or at risk for infection with a virus via a rote and in in amount effective for treating and/or preventing the disease, disorder, and/or condition associated with the viral infection in the subject. In some embodiments, the PTP4A3 inhibitor is 7-imino-2-phenylthieno[3,2-c]pyridine-4,6(5H,7H)-dione, thienopyridone, a pharmaceutically acceptable salt thereof, a prodrug thereof, a metabolite thereof, or any combination thereof. In some embodiments, the PTP4A3 inhibitor has the following general structure:

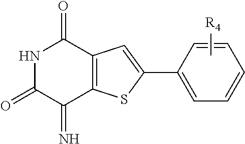

wherein:
R⁴ is selected from the group consisting of H, —OH,-OC₁a alkyl, —OR, trifluoroC₁₋₄alkoxy, —SC₁₋₄alkyl. —SR^{a}, —SO₂—C₁₋₄alkyl, —SO₂R^{a}, —SOC₁₋₄alkyl, —SOR^{a}, —SO₂NHR^{b}. —NR^{C}R^{d}, halo, —C₁₋₁₂ alkyl, —C₂₋₆alkenyl, —C₂₋₄alkynyl, —C₃₋₆ cycloalkyl, phenyl, benzyl, monocyclic heteroaryl optionally substituted with R^{b}, —C₃₋₆ cycoalkyl. —C₄₋₇ heterocycloalkyl containing one or two of O, S, and N, —OC(O)R^{b}, —OC(O)R^{b}, —P(O)(OR^{b})₁₋₂, —P(S)(OR^{b})₁₋₂, —P(O)(NR^{C}R^{d})₁₋₂, —P(S)(NR^{C}R^{d})₁₋₂, —O(CH₂—CH₂—O)₁₋₄CH₃, —CN, —NO₂, —C(O)C₁₋₄ alkyl, and —C(OH—R^{b},
R^{a} is selected from the group consisting of —C₃₋₆cycloalkyl, —C₂₋₆alkenyl, —C₂₋₆ alkynyl, phenyl, benzyl, and monocyclic heteroaryl, wherein the phenyl, benzyl, or monocyclic heteroaryl is optionally substituted with R^{b};
R^{b} in each instance is independently selected from the group consisting of H, halo, —OH, —COOH, —C₁₋₁₂alkyl, phenyl, benzyl, and monocyclic heteroaryl, wherein the phenyl, benzyl, or monocyclic heteroaryl is optionally substituted with —C₂₋₆alkenyl, —C₂₋₆ alkynyl, trifluroC₁₋₄ alkoxy, —OC₁₋₄ allyl, —O(CH₂—CH₂—O)₁₋₄ CH₃, —O-phenyl, —O— benzyl, —NC₁₋₄ alkyl, —N-phenyl, and —N-benzyl, or —N-monocyclic heteroaryl;
R^{C} and R^{d} are each independently selected from the group consisting of H, —C₁₋₄ alkyl, —C(O)—C₁₋₄ alkyl, —C(O)—R^{e}, —C₁₋₄ alkyl-R^{b}, —SO₂—R^{a}, —SO₂—C₁₋₄ alkyl, phenyl, benzyl, and monocyclic heteroaryl, wherein the phenyl, benzyl, or monocyclic heteroaryl is optionally substituted with R^{b}, or
or R^{C} and R^{d} taken together with the nitrogen to which they are attached represent an optionally substituted monocyclic heterocycloalkyl containing one or more of O, S, and N,
   and/or is a pharmaceutically acceptable salt thereof, prodrug thereof metabolite thereof, or ay combination thereof. In some embodiments, the disease, disorder, and/or condition associated with the viral infection comprises alveolar inflammation, alveolar thickening, neutrophil infiltration into the lung, endothelial barrier disruption, fibrosis, or any combination thereof.

In some embodiments, the presently disclosed subject matter also relates to methods for preventing ad/or treating chemical damage to a lung in a subject. In some embodiments, the methods comprise, consist essentially of, or consist of administering to a subject in need thereof an effective amount of a PTP4A3 inhibitor. In some embodiments, the chemical damage results in alveolar thickening, neutrophil infiltration, endothelial barrier disruption, fibrosis, or any combination thereof. In some embodiments, the PTP4A3 inhibitor is 7-imino-2-phenylthieno[3,2-c]pyridin-4,6(5H,7H)-dione, thienopyridone, pharmaceutically acceptable salts thereof, prodrugs thereof, metabolites thereof, or ay combination thereof. In some embodiments, the PTP4A3 inhibitor has the following general structure:

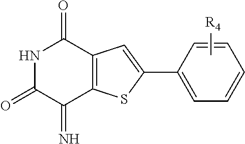

wherein:
R⁴ is selected from the group consisting of H, —OH, —OC₁₋₄alkyl, —OR, trifluoroC₁₋₄alkoxy, —SC₁₋₄ alkyl. —SR^{a}, —SO₂—C₁₋₄ alkyl, —SO₂R^{a}, —SOC₁₋₄alkyl, —SOR^{a}, —SO₂NHR^{a}, —NR^{C}R^{d}, halo, —C₁₋₁₂alkyl, —C₂₋₆alknyl, —C₂₋₄alkynyl, —C₃₋₆cycloalkyl, phenyl, benzyl, monocyclic heteroaryl optionally substituted with R^{b}, —C₃₋₆ cycloalkyl, —C₄₋₇ heterocycloalkyl containing one or two of O, S, and N, —OC(O)R^{b}, —OC(O)R^{b}, —P(O)(OR^{b})₁₋₂, —P(S)(OR^{b})₁₋₂, —P(O)(NR^{C}R^{d})₁₋₂, —P(S)(NR^{C}R^{d})₁₋₂, —O(CH₂—CH₂—O)₁₋₄CH₃, —CN. —NO₂, —C(O)C₁₋₄ alkyl, and —C(O)—R^{b},
R^{a} is selected from the group consisting of-C₆cycloalkyl. —C₂₋₆alkenyl. —C₂-₆alkynyl, phenyl, benzyl, and monocyclic heteroaryl, wherein the phenyl, benzyl, or monocyclic heteroaryl is optionally substituted with R^{b};
R^{b} in each instance is independently selected from the group consisting of H, halo, —OH. —COOH, —C₁₋₁₂alkyl, phenyl, benzyl, and monocyclic heteroaryl wherein the phenyl, benzyl, or monocyclic heteroaryl is optionally substituted with —CR alkenyl. —C₂₋₄ alkynyl, trifluroC₁₋₄ alkoxy, —OC₁₋₄ alkyl, —O(CH₂—CH₂—O)₁₋₄CH₃, —O-phenyl. —O— benzyl, —NC₁₋₄ alkyl, —N-phenyl, and —N-benzyl, or —N-monocyclic heteroaryl;
R^{C} and R^{d} are each independently selected from the group consisting of H. —C₁₋₄alkyl. —C(O)—C₁₋₄ alkyl, —C(O)—R^{e}, —C₁₋₄alkyl-R^{a}, —SO₂—R^{a}—SO₂—C₁₋₄ alkyl, phenyl, benzyl, and monocyclic heteroaryl, wherein the phenyl benzyl or monocyclic heteroaryl is optionally substituted with R^{b}, or
or R^{C} and R^{d} taken together with the nitrogen to which they are attached represent an optionally substituted monocyclic heterocycloalkyl containing one or more of O, S, and N, and/or is a pharmaceutically acceptable salt thereof, prodrug thereof, metabolite thereof, or any combination thereof. In some embodiments, the administering is oral, intravenous, intramuscular, subcutaneous, intraperitoneal, intranasal, pulmonary, or any combination thereof.

Accordingly, it is an object of the presently disclosed subject matter to provide compositions and methods for treating and/or preventing diseases, disorders, and/or conditions associated with viral infections and/or for treating and/or preventing chemical damage to lungs in subjects.

An object of the presently disclosed subject matter having been stated herein above, and which is achieved in whole or in part by the presently disclosed subject matter, other objects will become evident as the description proceeds when taken in connection with the accompanying Figures as best described herein below.

### BRIEF DESCRIPTION OF THE FIGURES

FIGS. 1A-1D. Exposure to subunit 1 of SARS-CoV-2 Spike protein (S1SP) induced weight loss and alveolar inflammation. (FIG. 1A) S1SP-instilled male K18-hACE2 transgenic mice (squares) exhibited weight loss compared to saline controls (circles). (FIGS. 1B-1D) As compared to saline controls (black squares), S1SP-instilled male K18-hACE2 transgenic mice (white squares) had elevated protein (FIG. 1B) and leukocyte (FIG. 1C) concentrations in BALF, especially in monocytes and neutrophils (FIG. 1D). Means±SEM; n=3; *: p<0.05; **: p<0.01; ***: p<0.001; ****: p<0.0001 with 2-way ANOVA followed by Tukey's (FIGS. 1A and 1D) or Student's t-test (FIGS. 1B and 1C).

FIG. 2. Exposure to the S1 subunit of SARS-CoV-2 Spike Protein induced alveolar “cytokine storm”. As compared to saline controls (black squares), S1SP-instilled male K18-hACE2 transgenic mice (white squares) experienced induced levels of IFNγ, IL-1β, IL-6, IL-17, MCP-1, KC, TNFα, and MIP1α. Means±SEM; n=3; *: p<0.05; **: p<0.01 with Mann-Whitney U.

FIGS. 3A-3D. The S1 subunit of the SARS-CoV-2 Spike Protein (S1SP) caused acute lung injury and the activation of the STAT3 and NFκB inflammatory pathways. (FIG. 3A) H&E staining of lung sections demonstrated septal thickening, neutrophil infiltration, and edema in S1SP-instilled mice (right panel) compared to saline controls (left panel). (FIGS. 3B-3D) Western blot analysis of lung homogenates revealed increased phosphorylation of IκBα and STAT3. Bands were normalized to 13-actin and are shown as fold of vehicle control. Means±SEM; n=3; *: p<0.05; with Student's t-test.

FIG. 4. The S1 subunit of SARS-CoV-2 spike protein S (S1SP) caused a concentration-dependent decrease in barrier function of human lung microvascular endothelial cells (HLMVEC), as measured by transendothelial electrical resistance (TER). Human lung microvascular endothelial cells were evaluated for barrier dysfunction after exposure to vehicle or S1SP. The barrier function of confluent endothelial cell monolayers was estimated using electric cell-substrate impedance sensing (ECIS) model 1600ζθ (applied Biophysics) as described (Catravas et al., 2010; Barabutis et al., 2018). Values are normalized to t=0. ****: p<0.0001 from Vehicle by 2-way ANOVA for repeated measures with Bonferroni's post-test. Means±SEM of 3-4/group. Time=hours.

FIG. 5. Cytokine content in mouse PTP4A3 wildtype (MsTu WT) and PTP4A3 knockout (MsTu KO) colon cancer cells as measured by Luminex. PTP4A3 knockout cells were evaluated for their ability to produce/secrete chemokines and cytokines (versus PTP4A3 WT cells). The cell lines are described in McQueeney et al., 2018). PTP4A3 knockout (MsTu KO) cells expressed lower levels of TNFα, LIF, MIP-1b, G-CSF, IL-6, and MIP-2 as compared to PTP4A3 wildtype (MsTu WT) cells. See Sims et al., 2013 for cell generation.

FIGS. 6A-6F. KVX-053/JMS-053 blocked Spike Protein-induced ALI in mice. K18-hACE2 mice received Vehicle or KVX-053/JMS-053 after exposure to the SARS-CoV-2 spike protein S1 subunit, (SP) it. SP increased BALF protein (FIG. 6A), WBC (FIG. 6B), and neutrophil (FIG. 6C) content, activated lung tissue STAT3 (FIG. 6D) and NFκB (FIG. 6E), and produced histological signs of hyaline membrane, alveolar (ALV) septal thickening and hypercellularity (FIG. 6F; H&E stain). Post-treatment with KVX-053 completely blocked (FIGS. 6A, 6D, and 6E) or significantly attenuated (FIGS. 6B, 6C, and 6F) all signs of SP-induced inflammation and ALI. Means±SEM of n=3 per group, ns: not significant, *: p<0.05, **: p<0.01, ***: p<0.001, ****: p<0.0001 with one-way ANOVA followed by Tukey's post-hoc test.

FIGS. 7A-7N. Exposure to the S1 subunit of SARS-CoV-2 Spike Protein (S1SP) induced alveolar “cytokine storm”, which was ameliorated by KVX-053/JMS-053 treatment. Different pro-inflammatory cytokines in mouse bronchial lavage fluid (BALF) were analyzed using a murine 32 plex assay. Pro-inflammatory cytokines included G-CSF (FIG. 7A), IFNγ (FIG. 7B), IL-12 (FIG. 7C), Eotaxin (FIG. 7D), IL-10 (FIG. 7E), IL-17 (FIG. 7F), GM-CSF (FIG. 7G), IL-6 (FIG. 7H), KC (FIG. 7I), MCP-1 (FIG. 7J), MIP-1a (FIG. 7K), RANTES (FIG. 7L), VEGF (FIG. 7M), and TNF-α (FIG. 7N). Results indicated that S1SP induced activation of multiple cytokines and chemokines in BALF. JMS-053 treatment was administered ip at 10 mg/kg. Means±SEM; n=3; *: p<0.05; **: p<0.01 with Mann-Whitney U. IT: intratracheal instillation.

FIGS. 8A and 8B. KVX-053 blocked Covid-19 spike protein (SP)-induced endothelial barrier disruption in human lung endothelial cells. HLMVEC were seeded on gold electrodes in a Electric Cell-substrate Impedance Sensing apparatus (ECIS), model 1600ζθ and let grow till confluency. When a stable resistance was achieved (>800Ω), HLMVEC were pre-treated with KVX-053 (12 μM) for 2 hours and half (150 minutes) and then exposed to S1SP 10 nM (FIG. 8A). Compared to vehicle pre-treatment, KVX-053 demonstrated to prevent S1 SP-induced endothelial barrier dysfunction. Similarly, when KVX-053 (25 μM) was administered after 4 hours of S1SP 10 nM exposure (post treatment), it showed to repair S1SP-induced hyperpermeability (FIG. 8B). Values are normalized to t=0. ****: p<0.0001 from Vehicle and/or control by 2-way ANOVA for repeated measures with Bonferroni's post-test. Means±SEM of 3-5/group.

FIGS. 9A-9D. KVX-053 blocks spike protein-induced proinflammatory chemokine release in vivo (i.e., Bronchial Alveolar Lung Fluid (BALF)). BALF was isolated 72 hours after spike protein instillation and KVX-053 treatment. BALF fluid was then evaluated for cytokine and chemokine levels via Luminex as described in Colunga Biancatelli et al., 2021. Shown are four chemokines that were decreased in expression after KVX-053 treatment: MCP-1/CCL2 (FIG. 9A), TNFα (FIG. 9B), MIP1α/CCL3 (FIG. 9C), and KC/CXCL1 (FIG. 9D). *: p<0.05; **: p<0.01; ***: p<0.001.

FIGS. 10A-10E. KVX-053/JMS-053 blocks chemical-induced lung injury in vivo. Lung injury was induced using the sulfur mustard analog CEES and the effects of KVX-053 were determined. (FIG. 10A) Histological evidence of injury (H&E). (FIG. 10B) alveolar proteinosis, WBC, and activation of ERK, 6 days post-instillation. (FIG. 10C) CEES elicited persistent increases in TNF-α, IL-6, infiltrative M2 macrophages (CD45⁺, CD11b⁺, CD68⁺). (FIG. 10D) activation of STAT3, AKT, and H2AX. (FIG. 10E) downward shift of Pressure-Volume Loops, increased Systemic and Newtonian resistances (Rrs and Rn), increased aSMA, 10 days post-instillation. KVX-053 (10 mg/kg/24 hours) prevented CEES-induced ALI (*: CEES vs VEH; #: CEES+JMS-053 vs CEES+VEH; #/*: p<0.05; ##/**: p<0.01; ***: p<0.001 with 1-way ANOVA and Tukey's; n=3-5).

### DETAILED DESCRIPTION

### I. General Considerations

Severe Acute Respiratory Syndrome coronavirus-2 (SARS-CoV-2) is a zoonotic beta coronavirus that is responsible for the current COVID-19 pandemic. As of Jan. 5, 2021, worldwide and in the US, respectively, >85.9 MM and >20.8 MM total confirmed COVID-19 cases and >1.9 MM and >354,000 COVID-19 deaths have been recorded. As with other respiratory conditions, COVID-19 disease manifestations can vary widely with the most well-known symptoms being associated with respiratory distress (e.g., dry cough, shortness of breath, difficulty breathing). While a majority of diagnosed/confirmed COVID-19 cases are mild, a subset of patients (˜5%) develop ARDS, septic shock and multi-organ failure, and about half of these patients will die. Importantly, pneumonia and ARDS typically occur late in the course of infection, between 5 and 10 days from the onset of symptoms (Rivellese & Prediletto, 2020).

The primary cause of death in COVID-19 patients is progressive respiratory failure (Ackermann et al., 2020), which is similar to that was observed during the 2003 SARS epidemic, caused by SARS-CoV-1, and is likely to occur with future coronavirus and influenza infections. Moreover, coronaviruses mutate readily, which can alter transmissibility, lethality and the efficacy of existing antibody and RNA therapies. The clinical worsening in the later phases of COVID-19 is likely not due to viral replication but rather due to the influx, through the pulmonary microvascular endothelium and epithelium, of neutrophils and monocytes-macrophages, which are the source of the pro-inflammatory cytokines, most notably IL-6 and TNFα. There is a significant need to identify safe and efficacious drugs that prevent or reverse the initial pulmonary damage and ARDS associated with COVID-19 and other viral infections. Such agents would provide an enormous benefit to millions of infected individuals now and for the foreseeable future.

SARS-CoV-2 binds to the pulmonary microvascular epithelium and endothelium (Jamilloux et al., 2020). SAR-CoV-2, like other coronaviruses, infects human airway cells by binding of its S (Spike) glycoprotein to the human angiotensin-converting enzyme 2 (hACE2) with S protein priming by the host serine protease TMPRSS2 (Hoffman et al., 2020). Expression of ACE2 and TMPRSS2 have been observed on both pulmonary microvascular epithelium and endothelium (Donoghue et al., 2000; Jamilloux et al., 2020). Previous studies with the closely related SARS-CoV-1 revealed that the Spike 1 (S1) protein can induce ER stress and cytokine release (Liu et al., 2007; Versteeg et al., 2007; Haga et al., 2008). Both the Spike S1 and S2 proteins as well as the Spike protein S1 subunit Receptor Binding Domain sequence disrupt cultured human brain endothelial barrier function and trigger a pro-inflammatory response (Buzhdygan et al., 2020). Our hypothesis is the Spike protein initiates the loss of pulmonary endothelial cell and epithelial barrier function, which leads to apoptosis and subsequent viral propagation in the cells lining the damaged respiratory tract triggering an immune response—usually a localized burst of inflammation with immune cell recruitment to engage and eliminate the virus (Jamilloux et al., 2020; Rivellese & Prediletto, 2020). While this localized immune response can fade with patient recovery, some patients develop widespread inflammation extending to all organs of the body (Shi et al., 2020). An uncontrolled immune response can trigger a systemic inflammatory response or “cytokine storm” during which the body attacks itself resulting in more damage (Chen et al., 2020; Shi et al., 2020). Collectively, these observations strongly justify efforts to protect and restore pulmonary vascular epithelial and endothelial barrier function after Spike protein exposure. Protein tyrosine phosphatase PTP4A3 (a/k/a PRL3) regulates vascular barrier function (McQueeney et al., 2017; Bersini et al., 2020) and the release of cytokines (Aguilar-Sopena et al., 2020). Thus, in some embodiments the presently disclosed subject matter relates to use of PTP4A3 inhibitors against SARS-CoV-2.

No current FDA-approved drugs treat the ALI and ARDS associated with COVID-19. COVID-19 clinical management strategies focus primarily on prevention and control measures used in conjunction with supportive care (i.e., supplemental oxygen and mechanical ventilation). The FDA has authorized the emergency use of remdesivir as it may be beneficial with respect to patient recovery time but it has not been shown conclusively to markedly increase patient survival. We also recognize there has been an enormous effort to develop vaccines, although they could have limited efficacy against future viral pandemics, including those mediated by coronaviruses. With respect to therapies directed toward the “cytokine storm”, anti-cytokine approaches targeting IL-1, IL-6, IFNγ, and IL-18 have shown some promise, albeit in diseases other than COVID-19 (Jamilloux et al., 2020; Shi et al., 2020; Sun et al., 2020). Therefore, in some embodiments the presently disclosed subject matter relates to novel agents that prevent and/or mitigate the pulmonary effects of SARS-CoV-2 and other viral infections as well as approaches to prevent and treat ARDS from other causes, including but not limited to chemical injuries/damage to the lungs.

### II. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a”, “an”, and “the” refer to “one or more” when used in this application, including in the claims. For example, the phrase “an antibody” refers to one or more antibodies, including a plurality of the same antibody. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

A disease or disorder is “alleviated” if the severity of a symptom of the disease, condition, or disorder, or the frequency at which such a symptom is experienced by a subject, or both, are reduced.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The terms “additional therapeutically active compound” and “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease, or disorder being treated.

As used herein, the term “adjuvant” refers to a substance that elicits an enhanced immune response when used in combination with a specific antigen.

As use herein, the terms “administration of” and/or “administering” a compound should be understood to refer to providing a compound of the presently disclosed subject matter to a subject in need of treatment.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition, and are encompassed within the nature of the phrase “consisting essentially of”.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

With respect to the terms “comprising”, “consisting of”, and “consisting essentially of”, where one of these three terms is used herein, the presently disclosed and claimed subject matter can include the use of either of the other two terms. For example, a composition that in some embodiments comprises a given active agent also in some embodiments can consist essentially of that same active agent, and indeed can in some embodiments consist of that same active agent.

As use herein, the terms “administration of” and or “administering” a compound should be understood to mean providing a compound of the presently disclosed subject matter or a prodrug of a compound of the presently disclosed subject matter to a subject in need of treatment.

The term “adult” as used herein, is meant to refer to any non-embryonic or non-juvenile subject. For example, the term “adult adipose tissue stem cell”, refers to an adipose stem cell, other than that obtained from an embryo or juvenile subject.

As used herein, an “agent” is meant to include something being contacted with a cell population to elicit an effect, such as a drug, a protein, a peptide. An “additional therapeutic agent” refers to a drug or other compound used to treat an illness and can include, for example, an antibiotic or a chemotherapeutic agent.

As used herein, an “agonist” is a composition of matter which, when administered to a mammal such as a human, enhances or extends a biological activity attributable to the level or presence of a target compound or molecule of interest in the mammal.

An “antagonist” is a composition of matter which when administered to a mammal such as a human, inhibits a biological activity attributable to the level or presence of a compound or molecule of interest in the mammal.

As used herein, “alleviating a disease or disorder symptom”, means reducing the severity of the symptom or the frequency with which such a symptom is experienced by a patient, or both.

As used herein, an “analog” of a chemical compound is a compound that, by way of example, resembles another in structure but is not necessarily an isomer (e.g., 5-fluorouracil is an analog of thymine).

As used herein, amino acids are represented by the full name thereof, by the three letter code corresponding thereto, and/or by the one-letter code corresponding thereto, as summarized in Table 1:

TABLE 1
Amino Acid Codes and Functionally Equivalent Codons

| Full Name | 3-Letter | 1-Letter | Functionally Equivalent Codons |
|---|---|---|---|
| Aspartic Acid | Asp | D | GAC; GAU |
| Glutamic Acid | Glu | E | GAA; GAG |
| Lysine | Lys | K | AAA; AAG |
| Arginine | Arg | R | AGA; AGG; CGA; CGC; CGG; CGU |
| Histidine | His | H | CAC; CAU |
| Tyrosine | Tyr | Y | UAC; UAU |
| Cysteine | Cys | C | UGC; UGU |
| Asparagine | Asn | N | AAC; AAU |
| Glutamine | Gln | Q | CAA; CAG |
| Serine | Ser | S | ACG; AGU; UCA; UCC; UCG; UCU |
| Threonine | Thr | T | ACA; ACC; ACG; ACU |
| Glycine | Gly | G | GGA; GGC; GGG; GGU |
| Alanine | Ala | A | GCA; GCC; GCG; GCU |
| Valine | Val | V | GUA; GUC; GUG; GUU |
| Leucine | Leu | L | UUA; UUG; CUA; CUC; CUG; CUU |
| Isoleucine | Ile | I | AUA; AUC; AUU |
| Methionine | Met | M | AUG |
| Proline | Pro | P | CCA; CCC; CCG; CCU |
| Phenylalanine | Phe | F | UUC; UUU |
| Tryptophan | Trp | W | UGG |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the compositions of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the compositions of the presently disclosed subject matter.

The term “amino acid” is used interchangeably with “amino acid residue”, and may refer to a free amino acid and to an amino acid residue of a peptide. It will be apparent from the context in which the term is used whether it refers to a free amino acid or a residue of a peptide.

Amino acids have the following general structure:

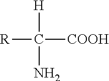

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

The term “antibody”, as used herein, refers to an immunoglobulin molecule which is able to specifically or selectively bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter may exist in a variety of forms. The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fᵥ, single chain F, (scFv), complementarity determining regions (CDRs), V_{L} (light chain variable region), V_{H} (heavy chain variable region), Fab, F(ab′)₂ and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce F(ab′)₂ a dimer of Fab which itself is a light chain joined to V_{H}—C_{H1} by a disulfide bond. The F(ab′)₂ may be reduced under mild conditions to break the disulfide linkage in the hinge region, thereby converting the F(ab′)₂ dimer into an Fab₁ monomer. The Fab₁ monomer is essentially an Fab with part of the hinge region. While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

An “antibody heavy chain”, as used herein, refers to the larger of the two types of polypeptide chains present in all antibody molecules.

An “antibody light chain”, as used herein, refers to the smaller of the two types of polypeptide chains present in all antibody molecules.

The term “single chain antibody” refers to an antibody wherein the genetic information encoding the functional fragments of the antibody are located in a single contiguous length of DNA. For a thorough description of single chain antibodies, see Bird et al., 1988; Huston et al., 1988).

By “small interfering RNAs (siRNAs)” is meant, inter alia, an isolated dsRNA molecule comprised of both a sense and an anti-sense strand. In some embodiments, it is greater than 10 nucleotides in length, siRNA also refers to a single transcript which has both the sense and complementary antisense sequences from the target gene, e.g., a hairpin, siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides. RNA interference is a commonly used method to regulate gene expression. This effect is often achieved by using small interfering RNA or short hairpin RNA (shRNA).

The term “humanized” refers to an antibody wherein the constant regions have at least about 80% or greater homology to human immunoglobulin. Additionally, some of the nonhuman, such as murine, variable region amino acid residues can be modified to contain amino acid residues of human origin. Humanized antibodies have been referred to as “reshaped” antibodies. Manipulation of the complementarity-determining regions (CDR) is a way of achieving humanized antibodies. See for example, Jones et al., 1986; Riechmann et al., 1988, both of which are incorporated by reference herein. For a review article concerning humanized antibodies, see Winter & Milstein, 1991, incorporated by reference herein. See also U.S. Pat. Nos. 4,816,567; 5,482,856; 6,479,284; 6,677,436; 7,060,808; 7,906,625; 8,398,980; 8,436,150; 8,796,439; and 10,253,111; and U.S. Patent Application Publication Nos. 2003/0017534, 2018/0298087, 2018/0312588, 2018/0346564, and 2019/0151448, each of which is incorporated by reference in its entirety.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the presently disclosed subject matter include, but are not limited to, phosphorothioate oligonucleotides and other modifications of oligonucleotides.

An “aptamer” is a compound that is selected in vitro to bind preferentially to another compound (for example, the identified proteins herein). Often, aptamers are nucleic acids or peptides because random sequences can be readily generated from nucleotides or amino acids (both naturally occurring or synthetically made) in large numbers but of course they need not be limited to these.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample”, as used herein, refers to samples obtained from a subject, including but not limited to skin, hair, tissue, blood, plasma, cells, sweat, and urine.

As used herein, the term “chemically conjugated”, or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to reactions as described herein. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarily between two nucleic acids (e.g., two DNA molecules). When a nucleotide position in both of the molecules is occupied by nucleotides normally capable of base pairing with each other at a given position, the nucleic acids are considered to be complementary to each other at this position. Thus, two nucleic acids are complementary to each other when a substantial number (in some embodiments at least 50%) of corresponding positions in each of the molecules are occupied by nucleotides that can base pair with each (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. By way of example and not limitation, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

A “compound”, as used herein, refers to a polypeptide, an isolated nucleic acid, or other agent used in the method of the presently disclosed subject matter.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a condition, disease, or disorder for which the test is being performed.

A “test” cell is a cell being examined.

As used herein, the term “conservative amino acid substitution” is defined herein as an amino acid exchange within one of the five groups summarized in Table 2:

TABLE 2
Exemplary Conservative Amino Acid Substitutions

| Group | Characteristics | Amino Acids |
|---|---|---|
| A. | Small aliphatic, nonpolar, or slightly | Ala, Ser, Thr, Pro, Gly |
| | polar residues | |
| B. | Polar, negatively charged residues | Asp, Asn, Glu, Gln |
| | and their amides | |
| C. | Polar, positively charged residues | His, Arg, Lys |
| D. | Large, aliphatic, nonpolar residues | Met Leu, Ile, Val, Cys |
| E. | Large, aromatic residues | Phe, Tyr, Trp |

A “pathoindicative” cell is a cell that, when present in a tissue, is an indication that the animal in which the tissue is located (or from which the tissue was obtained) is afflicted with a condition, disease, or disorder.

A “pathogenic” cell is a cell that, when present in a tissue, causes, or contributes to a condition, disease, or disorder in the animal in which the tissue is located (or from which the tissue was obtained).

A tissue “normally comprises” a cell if one or more of the cell are present in the tissue in an animal not afflicted with a condition, disease, or disorder.

As used herein, the terms “condition”, “disease condition”, “disease”, “disease state”, and “disorder” refer to physiological states in which diseased cells or cells of interest can be targeted with the compositions of the presently disclosed subject matter. In some embodiments, a disease is cancer, which in some embodiments comprises a solid tumor.

As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA, and mRNA) or a defined sequence of amino acids and the biological properties resulting therefrom. Thus, a gene encodes a protein if transcription and translation of an mRNA corresponding to or derived from that gene produces the protein in a cell or other biological system and/or an in vitro or ex vivo system. Both the coding strand, the nucleotide sequence of which is identical to the mRNA sequence (with the exception of uracil bases presented in the latter) and is usually provided in Sequence Listing, and the non-coding strand, used as the template for transcription of a gene or cDNA, can be referred to as encoding the protein or other product of that gene or cDNA.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein in some embodiments at least about 95% and in some embodiments at least about 99%, by weight, of the protein or peptide in the preparation is the particular protein or peptide.

A “fragment”, “segment”, or “subsequence” is a portion of an amino acid sequence, comprising at least one amino acid, or a portion of a nucleic acid sequence comprising at least one nucleotide. The terms “fragment”, “segment”, and “subsequence” are used interchangeably herein.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, in some embodiments, at least about 100 to about 200 nucleotides, in some embodiments, at least about 200 nucleotides to about 300 nucleotides, yet in some embodiments, at least about 300 to about 350, in some embodiments, at least about 350 nucleotides to about 500 nucleotides, yet in some embodiments, at least about 500 to about 600, in some embodiments, at least about 600 nucleotides to about 620 nucleotides, yet in some embodiments, at least about 620 to about 650, and most in some embodiments, the nucleic acid fragment will be greater than about 650 nucleotides in length. In the case of a shorter sequence, fragments are shorter.

As used herein, a “functional” biological molecule is a biological molecule in a form in which it exhibits a property by which it can be characterized. A functional enzyme, for example, is one that exhibits the characteristic catalytic activity by which the enzyme can be characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′-ATTGCC-5′ and 3′-TATGGC-5′ share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul, 1990a, modified as in Karlin & Altschul, 1993). This algorithm is incorporated into the NBLAST and XBLAST programs of Altschul et al., 1990a, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Altschul et al., 1997) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarily between the nucleic acids, stringency of the conditions involved, the length of the formed hybrid, and the G:C ratio within the nucleic acids.

The term “ingredient” refers to any compound, whether of chemical or biological origin, that can be used in cell culture media to maintain or promote the proliferation, survival, or differentiation of cells. The terms “component”, “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins, and the like. Other ingredients that promote or maintain cultivation of cells ex vivo can be selected by those of skill in the art, in accordance with the particular need.

As used herein “injecting”, “applying”, and administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

Used interchangeably herein are the terms: 1) “isolate” and “select”; and 2) “detect” and “identify”.

The term “isolated”, when used in reference to compositions and cells, refers to a particular composition or cell of interest, or population of cells of interest, at least partially isolated from other cell types or other cellular material with which it naturally occurs in the tissue of origin. A composition or cell sample is “substantially pure” when it is at least 60%, or at least 75%, or at least 90%, and, in certain cases, at least 99% free of materials, compositions, cells other than composition or cells of interest. Purity can be measured by any appropriate method, for example, by fluorescence-activated cell sorting (FACS), or other assays which distinguish cell types. Representative isolation techniques are disclosed herein for antibodies and fragments thereof.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically or selectively binds to a target compound. A ligand (e.g., an antibody) “specifically binds to”, “is specifically immunoreactive with”, “having a selective binding activity”, “selectively binds to” or “is selectively immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically or selectively binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow & Lane, 1988, for a description of immunoassay formats and conditions that can be used to determine specific immunoreactivity.

A “receptor” is a compound that specifically or selectively binds to a ligand.

A ligand or a receptor (e.g., an antibody) “specifically binds to”, “is specifically immunoreactive with”, “having a selective binding activity”, “selectively binds to” or “is selectively immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically or selectively binds under hybridization conditions to a compound polynucleotide comprising a complementary sequence; an antibody specifically or selectively binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow & Lane, 1988 for a description of immunoassay formats and conditions that can be used to determine specific or selective immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to through ionic or hydrogen bonds or van der Waals interactions.

The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “modulate”, as used herein, refers to changing the level of an activity, function, or process. The term “modulate” encompasses both inhibiting and stimulating an activity, function, or process. The term “modulate” is used interchangeably with the term “regulate” herein.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double-stranded DNA and cDNA. Furthermore, the terms, “nucleic acid”, “DNA”, “RNA” and similar terms also include nucleic acid analogs, i.e. analogs having other than a phosphodiester backbone. For example, the so-called “peptide nucleic acids”, which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” refers to non-naturally occurring peptides or polypeptides. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

A “preventive” or “prophylactic” treatment is a treatment administered to a subject who does not exhibit signs, or exhibits only early signs, of a condition, disease, or disorder. A prophylactic or preventative treatment is administered for the purpose of decreasing the risk of developing pathology associated with developing the condition, disease, or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is in some embodiments greater than 90% pure, that is in some embodiments greater than 95% pure, and that is in some embodiments greater than 98% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

As used herein, the term “secondary antibody” refers to an antibody that binds to the constant region of another antibody (the primary antibody).

As used herein, the term “single chain variable fragment” (scFv) refers to a single chain antibody fragment comprised of a heavy and light chain linked by a peptide linker. In some cases scFv are expressed on the surface of an engineered cell, for the purpose of selecting particular scFv that bind to an antigen of interest.

As used herein, the term “mammal” refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The term “subject” as used herein refers to a member of species for which treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichthyes (bony fish), Amphibia (amphibians), Reptilia (reptiles), Ayes (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

As used herein, “substantially homologous amino acid sequences” includes those amino acid sequences which have at least about 95% homology, in some embodiments at least about 96% homology, more in some embodiments at least about 97% homology, in some embodiments at least about 98% homology, and most in some embodiments at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; in some embodiments in 7% (SDS), 0.5 M NaPO₄, 1 mM EDTA at 50° C. with washing in 1×SSC, 0.1% SDS at 50° C.; in some embodiments 7% SDS, 0.5 M NaPO₄, 1 mM EDTA at 50° C. with washing in 0.5×SSC, 0.1% SDS at 50° C.; and more in some embodiments in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990a; Altschul et al., 1990b; Altschul et al., 1997). The default settings provided with these programs are suitable for determining substantial similarity of nucleic acid sequences for purposes of the presently disclosed subject matter.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, in some embodiments, humans.

As used herein, a “subject in need thereof” is a patient, animal, mammal, or human, who will benefit from the method of this presently disclosed subject matter.

The term “substantially pure” describes a compound, e.g., a protein or polypeptide, which has been separated from components which naturally accompany it. Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

As used herein, the terms “vector”, “cloning vector”, and “expression vector” refer to a vehicle by which a polynucleotide sequence (e.g., a foreign gene) can be introduced into a host cell, so as to transduce and/or transform the host cell in order to promote expression (e.g., transcription and translation) of the introduced sequence. Vectors include plasmids, phages, viruses, etc.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

### III. Compositions Comprising PTP4A3 Inhibitors and Methods and Uses Thereof

As disclosed herein, the presently disclosed subject matter relates in some embodiments to composition comprising, consisting essentially of, or consisting of inhibitors of PTP4A3 biological activities (referred to herein as “PTP4A3 inhibitors”) and methods for using the same to inhibit PTP4A3 biological activities in cells, tissues, organs, and subjects.

As used herein, the phrase “PTP4A3” refers to the protein tyrosine phosphatase 4A3 genetic locus, its gene products, and the biological activities that its gene products produce. In humans, the PTP4A3 locus is present on chromosome 8, and multiple isoforms of transcription products are known to be generated from the human locus, including Accession Nos. NM 032611.3 (isoform 1; SEQ ID NO: 1) and NM 007079.3 (isoform 2; SEQ ID NO: 3) of the GENBANK® biosequence database. Accession Nos. NM_032611.3 and NM_007079.3 of the GENBANK® biosequence database encode protein products that have the amino acid sequences set forth in Accession Nos. NP_116000.1 (SEQ ID NO: 2) and NP_009010.2 (SEQ ID NO: 4) of the GENBANK® biosequence database, respectively.

Any PTP4A3 inhibitor can be used in the compositions and methods of the presently disclosed subject matter. Exemplary PTP4A3 inhibitors include small molecule inhibitors, anti-PTP4A3 antibodies and fragments and derivatives thereof that bind to PTP4A3 polypeptides to inhibit their biological activities, and nucleic acid-based PTP4A3 inhibitors that bind to PTP4A3 nucleic acids to inhibit their activities.

With respect to small molecule inhibitors of PTP4A3, various examples of small molecule inhibitors of PTP4A3 are known, such as but not limited to those described in U.S. Pat. No. 10,308,663, which is incorporated herein by reference in its entirety. As set forth therein, exemplary PTP4A3 inhibitors include small molecules having the following general structure:

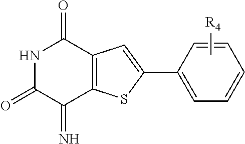

wherein
R⁴ is selected from the group consisting of H, —OH, —OC₁₋₄alkyl, —OR^{a}, trifluoroC₁₋₄ alkoxy. —SC₁₋₄alkyl, —SR^{a}, —SO₂—C₁₋₄allyl, —SO₂R^{a}, —SOC₁₋₄alkyl. —SOR^{a}, —SO₂NHR^{a}, —NR^{C}R^{d}, halo. —C₁₋₁₂alkyl. —C₂₋₆alkenyl, —C₂₋₆alkynyl, —C₃₋₆cycloalkyl, phenyl, benzyl, monocyclic heteroaryl optionally substituted with R^{b}, —C₃₋₆ cycloalkyl, —C₄₋₇heterocycloalkyl containing one or two of O, S, and N. —OC(O)R^{b}, —OC(O)R^{b}, —P(O)(OR^{b})₁₋₂, —P(S)(OR)₁₋₂, —P(O)(NR^{C}R^{d})₁₋₂, —P(S)(NR^{C}R^{d})₁₋₂, —O(CH₂—CH₂—O)₁₋₄CH₃, —CN. —NO₂, —C(O)C₁₋₄alkyl, and —C(O)—R^{b}.
R^{a} is selected from the group consisting of —C₃₋₆ cycloalkyl, —C₂₋₆alkenyl, —C₂₋₆ alkynyl, phenyl, benzyl, and monocyclic heteraryl, wherein the phenyl, benzyl, or monocyclic heteroaryl is optionally substituted with R^{b};
R^{b} in each instance is independently selected from the group consisting of H, halo. —OH, —COOH. —C₁₋₁₂ alkyl, phenyl, benzyl, and monocyclic heteroayl, wherein the phenyl, benzyl, or monocyclic heteroaryl is optionally substituted with —C₂₋₆alkenyl, —C₂₋₆alkynyl, trifluroC₁₋₄alkoxy, —OC₁₋₄ alkyl. —O(CH₂—CH₂—O)₁₋₄CH₃, —O-phenyl. —O— benzyl. —NC₁₋₄alkyl. —N-phenyl, and —N-benzyl or —N-monocyclic heteroaryl:
R^{C} and R^{d} are each independently selected from the group consisting of H, —C₁₋₄alkyl, —C(O)—C₁₋₄alkyl, —C(O)—R^{e}, —C₁₋₄alkyl-R^{e}, —SO₂—R^{a}. —C₁₋₄alkyl, phenyl, benzyl, and monocyclic heteroaryl, wherein the phenyl, benzyl, or monocyclic heteroaryl is optionally substituted with R^{b}.
or R^{C} and R^{d} taken together with the nitrogen to which they are attached represent an optionally substituted monocyclic heterocycloalkyl containing one or more of O, S, and N.

A particular example of PTP4A3 inhibitors that falls within the scope of this structure include, but are not limited to KVX-053 (also called 7-imino-2-phenylthieno[3,2-c]pyridine-4,6(5H,7H)-dione, JMS-631-053, and JMS-053; CAS No. 1954650-11-3), which has the following structure:

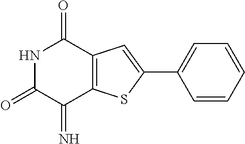 [SMILES: N=C1C(=O)NC(=O)c2cc(-c3ccccc3)sc21]

Thus, in some embodiments the presently disclosed subject matter provides the PTP4A inhibitor, KVX-053/JMS-053 for use to prevent or repair viral protein-mediated ALI due to viral infection including but not limited to SARS-CoV-2 infection. PTP4A regulates vascular permeability and cytokine release (McQueeney et al., 2017; Castro-Sanchez et al., 2018; Aguilar-Sopena et al., 2020; Bersini et al., 2020). None of the current COVID-19 clinical management strategies target protein tyrosine phosphatases, more specifically PTP4A3. Indeed, there are not yet any FDA-approved tyrosine phosphatase inhibitors. Moreover, there have been almost no meaningful efforts focused on the central role of ALI in the pathogenesis of SARS-CoV-2. As our Preliminary Data strongly suggest, we believe the application of a potent, selective, allosteric, and drug-like PTP4A inhibitor to reduce the pulmonary barrier damage caused by the Spike protein and the resulting cellular infiltrate and cytokine release could be a highly valuable therapeutic intervention for SARS-CoV-2 and future coronavirus and influenza pandemics.

Additional PTP4A3 inhibitors include those described in U.S. Patent Application Publication No. 2022/0017534, which is also incorporated by reference herein in its entirety. Exemplary PTP4A3 inhibitors thus also include, but are not limited to:

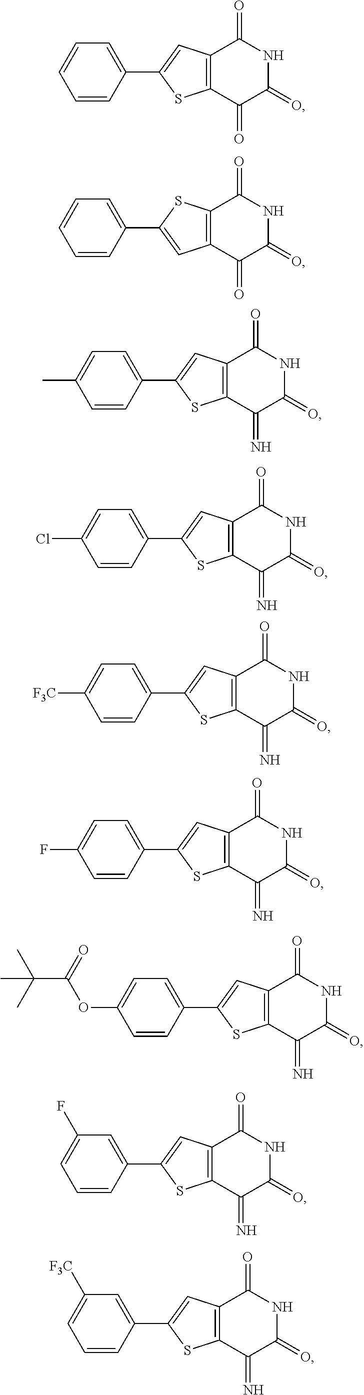

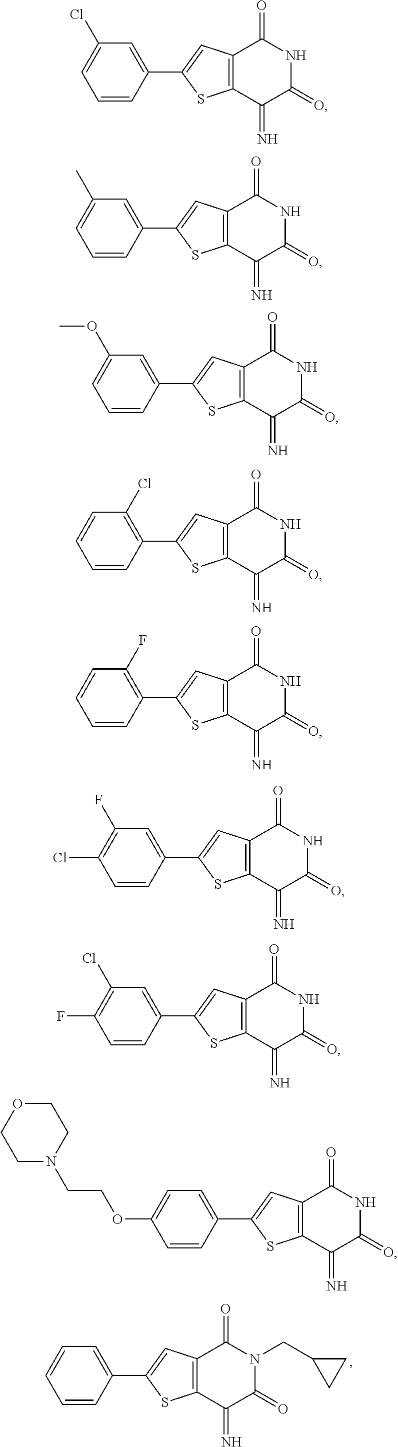

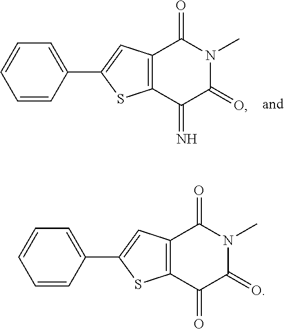 [SMILES: CN1C(=O)C(=N)c2sc(-c3ccccc3)cc2C1=O ; CN1C(=O)C(=O)c2sc(-c3ccccc3)cc2C1=O]

ALI leading to ARDS is the major cause of COVID-19 lethality. Frequently, signs of ALI appear prior to significant viral replication suggesting causation by elements other than the intact virus. We have developed an ALI model of COVID-19 by intratracheally instilling the S1 subunit of SARS-CoV-2 Spike Protein in K18-hACE2 transgenic mice that overexpress the human receptor ACE2 and investigated outcomes 72 hours later. Mice exhibited an acute decline in body weight, dramatically increased white blood cell and protein concentrations in bronchoalveolar lavage fluid (BALF) compared to saline-instilled controls. This inflammatory reaction also included huge increases in BALF levels of multiple cytokines. Histological examination of fixed lung sections revealed hyaline membranes, alveolar septal thickening, and neutrophil infiltrates. Molecular analysis of lung homogenates demonstrated activation of STAT3 and NFκB pathways, as reflected in increased phosphorylation of STAT3 and IKBα. We conclude that the intratracheal instillation of the S1 subunit SARS-CoV-2 Spike Protein in K18-hACE2 transgenic mice provides a valid model of COVID-19-induced ALI, improving our understanding of SARS-CoV-2 induced lung injury and assisting in the investigation of new therapeutic approaches for the management of COVID-19 and other coronaviruses.

Thus, the presently disclosed subject matter provides a model of intratracheal instillation of the ACE2-binding S1 subunit of the SARS-CoV-2 Spike-protein (S1SP) in K18-hACE2 transgenic mice, which has been used to study the morphological, cellular, and molecular characteristics of the acute response.

Also, an aspect of the presently disclosed subject matter is that PTP4A3 inhibitors can be useful against viral infection including but not limited to SARS-CoV-2 infection and/or infection with an influenza A virus such as but not limited to H1N1. This is supported by strong data showing that Spike protein causes ARDS-like symptoms in mice, which are effectively blocked by an exemplary PTP4A3 inhibitor. In some embodiments, the presently disclosed subject matter provides the PTP4A inhibitor, KVX-053, for use to prevent or repair viral protein-mediated ALI due to SARS-CoV-2 infection. KVX-053 is also referred to herein as JMS-053.

Accordingly, the presently disclosed subject matter relates in some embodiments to methods for treating and/or preventing a disease, disorder, and/or condition associated with viral infection in a subject, the method comprising, consisting essentially of, or consisting of administering to the subject an effective amount of a protein tyrosine phosphatase 4A3 (PTP4A3) inhibitor. In some embodiments, the disease, disorder, and/or condition comprises lung damage, which can optionally be ALI and/or ARDS. In some embodiments, the PTP4A3 inhibitor is selected from the group consisting of 7-imino-2-phenylthieno[3,2-c]pyridine-4,6(5H,7H)-dione, thienopyridone, and compounds comprising the following general structure:

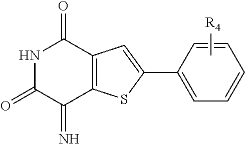

wherein
R⁴ is selected from the group consisting of H, —OH. —OC₁₋₄ alkyl, —OR^{a}, trifluoroC₁₋₄ alkoxy, —SC₁₋₄alkyl, —SR^{a}, —SO₂—C₁₋₄ alkyl. —SO₂R^{a}. —SOC₁₋₄ alkyl, —SOR^{a}, —SO₂NHR^{b}, —NR^{C}R^{d}, halo. —C₁₋₁₂alkyl, —C₂₋₆alkenyl. —C₂₋₆ alkynyl, —C₃₋₆cycloalkyl, phenyl, benzyl, monocyclic heteroaryl optionally substituted with R^{b}, —C₃₋₆ cycloalkyl, —C₄₋₇ heterocycloalkyl containing one or two of O, S, and N, —OC(O)R^{b}, —OC(O)R^{b}, —P(O)(OR^{b})₁₋₂, —P(S)OR^{b})₁₋₂. —P(O)(NR^{C}R^{d})₁₋₂, —P(S)(NR^{C}R^{d})₁₋₂. —O(CH₂—CH₂—O)₁₋₄CH₃, —CN. —NO₂, —C(O)C₁₋₄alkyl, and —C(O)—R^{b},
R^{a} is selected from the group consisting of-C₆cycloalkyl.-C₂alkenyl. —C₂₋₆ alkynyl, phenyl, benzyl, and monocyclic heteroaryl, wherein the phenyl, benzyl, or monocyclic heteroaryl is optionally substituted with R^{b};
R^{b} in each instance is independently selected from the group consisting of H, halo, —OH. —COOH, —C₁₋₁₂alkyl, phenyl, benzyl, and monocyclic heteroaryl, wherein the phenyl, benzyl, or monocylic heteroaryl is optionally substituted with —C₂₋₆alkenyl. —C₂₋₆alkynyl, trifluroC₁₋₄ alkoxy, —OC₁₋₄alkyl, —O(CH₂—CH₂—O)₁₋₄CH₃, —O-phenyl, —O—benzyl, —NC₁₋₄alkyl, —N-phenyl, and —N-benzyl, or —N-monocyclic heteroaryl,
R^{C} and R^{d} are each independently selected from the group consisting of H. —C₁₋₄ alkyl. —C(O)—C₁₋₄alkyl, —C(O)—R^{e}, —C₁₋₄ alkyl-R^{e}, —SO₂—R^{a}, —SO₂—C₁₋₄ alkyl, phenyl, benzyl, and monocyclic heteroaryl, wherein the phenyl, benzyl or monocyclic heteroaryl is optionally substituted with R^{b}, or
or R^{C} and R^{d} taken together with the nitrogen to which they are attached represent an optionally substituted monocyclic heterocycloalkyl containing one or more of O, S, and N,
   pharmaceutically acceptable salts thereof, prodrugs thereof, metabolites thereof, or any combination thereof.

Infection by any virus that causes lung injury can be treated with the compositions and methods of the presently disclosed subject matter. In some embodiments, the viral infection is a SARS-CoV-2 infection and/or an influenza A infection, optionally an infection with an H1N1 subtype of influenza A.

The presently disclosed subject matter also provides methods for reducing or inhibiting virus-induced alveolar inflammation and/or damage by administering to a subject in need thereof an effective amount of a PTP4A3 inhibitor. Any PTP4A3 inhibitor, including but not limited to the PTP4A3 inhibitors disclosed herein can be employed in the instant methods.

The presently disclosed subject matter also provides methods for reducing or inhibiting induction of an inflammatory cytokine and/or chemokine by a viral infection in a subject, optionally a SARS-CoV-2 infection, the method comprising administering to a subject in need thereof an effective amount of a PTP4A3 inhibitor. Any PTP4A3 inhibitor, including but not limited to the PTP4A3 inhibitors disclosed herein can also be employed in the instant methods. In some embodiments, the inflammatory cytokine and/or chemokine is selected from the group consisting of IFNγ, IL-13, IL-6, IL-17, MCP-1, KC, TNFα, MIP-1α, CLL2, CCL3, and CXCL1.

The presently disclosed subject matter also provides methods for reducing or inhibiting a pulmonary disease, disorder, and/or condition associated with a viral infection in a subject, or pulmonary damage resulting therefrom by administering to a subject in need thereof an effective amount of a PTP4A3 inhibitor. Any PTP4A3 inhibitor, including but not limited to the PTP4A3 inhibitors disclosed herein can also be employed in the instant methods. In some embodiments, the pulmonary disease, disorder, and/or condition comprises alveolar thickening, neutrophil infiltration, endothelial barrier disruption, fibrosis, or any combination thereof. In some embodiments, the viral infection is a SARS-CoV-2 infection and/or an influenza A infection, optionally an infection with an H1N1 subtype of influenza A.

The presently disclosed subject matter also provides for uses of PTP4A3 inhibitors for treating and/or preventing a disease, disorder, and/or condition associated with viral infection in a subject, and/or pulmonary damage resulting therefrom. In some embodiments, the disease, disorder, and/or condition associated with the viral infection is selected from the group consisting of ALI and ARDS. With respect to the presently disclosed uses, any PTP4A3 inhibitor, including but not limited to the PTP4A3 inhibitors disclosed herein can also be employed. In some embodiments, the PTP4A3 inhibitor is selected from the group consisting of 7-imino-2-phenylthieno[3,2-c]pyridine-4,6(5H,7H)-dione, thienopyridone, and compounds comprising the following general structure:

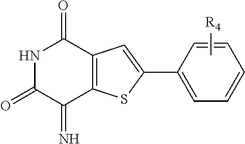

wherein:
R⁴ is selected from the group consisting of H, —OH, —OC₁₋₄ alkyl, —OR, trifluoroC1-₄alkoxy, —SC₁₋₄ alkyl. —SR^{a}. —SO₂—C₁₋₄ alkyl, —SO₂R^{a}, —SOC₁₋₄alkyl, —SOR^{a}, —SO₂NHR^{b}. —NR^{C}R^{d}, halo, —C₁₋₁₂alkyl, —C₂₋₆alkenyl, —C₂₋₆alkynyl, —C₃₋₆ cycloalkyl, phenyl, benzyl, monocyclic heteroaryl optionally substituted with R^{b}. —C₃₋₆cycloalkyl, —C₄₋₇ heterocycloalkyl containing one or two of O, S, and N, —OC(O)R^{b}. —OC(O)R^{b}, —P(O)(OR^{b})₁₋₂. —P(S)OR^{b})₁₋₂. —P(O)(NR^{C}R^{d})₁₋₂, —P(S)(NR^{C}R^{a})₁₋₂, —O(CH₂—CH₂—O)₁₋₄CH₃, —CN, —NO₂, —C(O)C₁₋₄alkyl, and —C(O)—R^{b},
R^{a} is selected from the group consisting of —C₃₋₆cycloalkyl, —C₂₋₆alkenyl, —C₂-₆alkynyl, phenyl, benzyl, and monocyclic heteroaryl, wherein the phenyl, benzyl, or monocyclic heteroaryl is optionally substituted with R^{b};
R^{b} in each instance is independently selected from the group consisting of H, halo. —OH, —COOH, —C₁₋₁₂alkyl, phenyl, benzyl, and monocyclic heteroaryl, wherein the phenyl, benzyl, or monocyclic heteroaryl is optionally substituted with —C₂₋₆ alkenyl, —C₂₋₆ alkynyl, trifluroC₁₋₄ alkoxy. —OC₁₋₄alkyl, —O(CH₂—CH₂—O)₁₋₄CH₃, —O-phemyl, —O— benzyl, —NC₁₋₄alkyl, —N-phenyl, and —N-benzyl, or —N-monocyclic heteroaryl:
R^{C} and R^{d} are each independently selected from the group consisting of H, —C₁₋₄alkyl, —C(O)—C₁₋₄alkyl, —C(O)—R^{e}, —C₁₋₄alkyl-R^{a}. —SO₂—R^{a}, —SO₂—C₁₋₄ alkyl, phenyl, benzyl, and monocyclic heteroaryl, wherein the phenyl, benzyl, or monocyclic heteroaryl is optionally substituted with R^{b}, or
or R^{C} and R^{d} taken together with the nitrogen to which they are attached represent an optionally substituted monocyclic heterocycloalkyl containing one or more of O, S, and N,
   pharmaceutically acceptable salts thereof, prodrugs thereof, metabolites thereof, or any combination thereof.

The presently disclosed subject matter also provides compositions comprising one or more PTP4A3 inhibitors for use in methods for treating and/or preventing a disease, disorder, and/or condition associated with viral infection in a subject, optionally a SARS-CoV-2 infection in a subject, by administering to a subject infected with or at risk for infection with a virus via a route and in an amount effective for treating and/or preventing the disease, disorder, and/or condition associated with the viral infection in the subject. In some embodiments, the disease, disorder, and/or condition associated with the viral infection comprises alveolar inflammation, alveolar thickening, neutrophil infiltration into the lung, endothelial barrier disruption, fibrosis, or any combination thereof.

In addition to lung damage resulting from viral infections, the presently disclosed subject matter also provides methods for preventing and/or treating chemical damage to the lungs of subjects. In some embodiments, the methods comprise, consist essentially of, or consist of administering to a subject in need thereof an effective amount of a PTP4A3 inhibitor as disclosed herein. In some embodiments, the chemical damage results in alveolar thickening, neutrophil infiltration, endothelial barrier disruption, fibrosis, or any combination thereof. In some embodiments, the PTP4A3 inhibitor is selected from the group consisting of 7-imino-2-phenylthieno[3,2-c]pyridine-4,6(5H,7H)-dione, thienopyridone, and compounds comprising the following general structure:

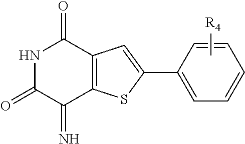

wherein:
R⁴ is selected from the group consisting of H, —OH. —OC₁₋₄ alkyl, —OR^{a}, trifluoroC₁₋₄ alkoxy, —SC₁₋₄alkyl, —SR^{a}, —SO₂—C₁₋₄ alkyl. —SO₂R^{a}. —SOC₁₋₄ alkyl, —SOR^{a}, —SO₂NHR^{b}, —NR^{C}R^{d}, halo. —C₁₋₁₂ alkyl, —C₂₋₆alkenyl. —C₂₋₆ alkynyl, —C₃₋₆cycloalkyl, phenyl, benzyl, monocyclic heteroaryl optionally substituted with R^{b}. —C₃₋₆cycloalkyl, —C₄₋₇ heterocycloalkyl containing one or two of O, S, and N, —OC(O)R^{b}. —OC(O)R^{b}, —P(O)(OR^{b})₁₋₂. —P(S)OR^{b})₁₋₂. —P(O)(NR^{C}R^{d})₁₋₂, —P(S)(NR^{C}R^{a})₁₋₂, —O(CH₂—CH₂—O)₁₋₄CH₃, —CN, —NO₂, —C(O)C₁₋₄alkyl, and —C(O)—R^{b},
R^{a} is selected from the group consisting of —C₃₋₆cycloalkyl, —C₂₋₆alkenyl, —C₂-₆alkynyl, phenyl, benzyl, and monocyclic heteroaryl, wherein the phenyl, benzyl, or monocyclic heteroaryl is optionally substituted with R^{b};
R^{b} in each instance is independently selected from the group consisting of H, halo. —OH, —COOH, —C₁₋₁₂alkyl, phenyl, benzyl, and monocyclic heteroaryl, wherein the phenyl, benzyl, or monocyclic heteroaryl is optionally substituted with —C₂₋₆ alkenyl, —C₂₋₆ alkynyl, trifluroC₁₋₄ alkoxy. —OC₁₋₄alkyl, —O(CH₂—CH₂—O)₁₋₄CH₃, —O-phemyl, —O— benzyl, —NC₁₋₄alkyl, —N-phenyl, and —N-benzyl, or —N-monocyclic heteroaryl:
R^{C} and R^{d} are each independently selected from the group consisting of H, —C₁₋₄alkyl, —C(O)—C₁₋₄alkyl, —C(O)—R^{e}, —C₁₋₄alkyl-R^{a}. —SO₂—R^{a}, —SO₂—C₁₋₄ alkyl, phenyl, benzyl, and monocyclic heteroaryl, wherein the phenyl, benzyl, or monocyclic heteroaryl is optionally substituted with R^{b}, or
or R^{C} and R^{d} taken together with the nitrogen to which they are attached represent an optionally substituted monocyclic heterocycloalkyl containing one or more of O, S, and N,
   pharmaceutically acceptable salts thereof, prodrugs thereof, metabolites thereof, or any combination thereof. In some embodiments, the administering is oral, intravenous, intramuscular, subcutaneous, intraperitoneal, intranasal, pulmonary, or any combination thereof.

### III.A. Formulations

Compositions as described herein comprise in some embodiments a composition that includes a pharmaceutically acceptable carrier. Suitable formulations include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents. In some embodiments, a formulation of the presently disclosed subject matter comprises an adjuvant, optionally an oil-based adjuvant.

The compositions used in the methods can take such forms as suspensions, solutions, or emulsions in oily or aqueous vehicles, and can contain formulatory agents such as suspending, stabilizing, and/or dispersing agents. The compositions used in the methods can take forms including, but not limited to perioral, intravenous, intraperitoneal, intramuscular, and intratumoral formulations. Alternatively or in addition, the active ingredient can be in powder form for constitution with a suitable vehicle (e.g., sterile pyrogen-free water) before use.

The formulations can be presented in unit-dose or multi-dose containers, for example sealed ampules and vials, and can be stored in a frozen or freeze-dried (lyophilized) condition requiring only the addition of sterile liquid carrier immediately prior to use.

For oral administration, the compositions can take the form of, for example, tablets or capsules prepared by a conventional technique with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulfate). The tablets can be coated by methods known in the art. For example, a neuroactive steroid can be formulated in combination with hydrochlorothiazide, and as a pH stabilized core having an enteric or delayed-release coating which protects the neuroactive steroid until it reaches the colon.

Liquid preparations for oral administration can take the form of, for example, solutions, syrups, or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring, and sweetening agents as appropriate. Preparations for oral administration can be suitably formulated to give controlled release of the active compound. For buccal administration, the compositions can take the form of tablets or lozenges formulated in conventional manner.

The compounds can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt).

The compounds can also be formulated in oils that are administered as water-in-oil emulsions, oil-in-water emulsions, or water-in-oil-in water emulsions.

The compounds can also be formulated in rectal compositions (e.g., suppositories or retention enemas containing conventional suppository bases such as cocoa butter or other glycerides), creams or lotions, or transdermal patches.

In some embodiments, the presently disclosed subject matter employs a composition that is pharmaceutically acceptable for use in humans. One of ordinary skill in the art understands the nature of those components that can be present in such a composition that is pharmaceutically acceptable for use in humans and also what components should be excluded from compositions that are pharmaceutically acceptable for use in humans.

### III.B. Doses

As used herein, the phrases “treatment effective amount”, “therapeutically effective amount”, “treatment amount”, and “effective amount” are used interchangeably and refer to an amount of a therapeutic composition sufficient to produce a measurable response (e.g., a biologically or clinically relevant response in a subject being treated). Actual dosage levels of active ingredients in the pharmaceutical compositions of the presently disclosed subject matter can be varied so as to administer an amount of the active compound(s) that is effective to achieve the desired therapeutic response for a particular subject. The selected dosage level can depend upon the activity of the therapeutic composition, the route of administration, combination with other drugs or treatments, the severity of the condition being treated, the condition and prior medical history of the subject being treated, etc. However, it is within the skill of the art to start doses of the compound at levels lower than required to achieve the desired therapeutic effect and to gradually increase the dosage until the desired effect is achieved.

The potency of a therapeutic composition can vary, and therefore a “therapeutically effective amount” can vary. However, one skilled in the art can readily assess the potency and efficacy of a candidate modulator of the presently disclosed subject matter and adjust the therapeutic regimen accordingly.

After review of the disclosure herein of the presently disclosed subject matter, one of ordinary skill in the art can tailor the dosages to an individual subject, taking into account the particular formulation, method of administration to be used with the composition, and other factors. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

For administration of a composition as disclosed herein, conventional methods of extrapolating human dosage based on doses administered to a murine animal model can be carried out using techniques known to one of ordinary skill in the art. Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich et al., 1966. Briefly, to express a mg/kg dose in any given species as the equivalent mg/m2 dose, multiply the dose by the appropriate km factor. In an adult human, 100 mg/kg is equivalent to 100 mg/kg×37 kg/m²=3700 mg/m².

For additional guidance regarding formulations and doses, see U.S. Pat. Nos. 5,326,902; 5,234,933; PCT International Publication No. WO 93/25521; Remington et al., 1975; Goodman et al., 1996; Berkow et al., 1997; Speight & Holford, 1997; Ebadi, 1998; Duch et al., 1998; Katzung, 2001; Gerbino, 2005.

### III.C. Routes of Administration

The presently disclosed compositions can be administered to a subject in any form and/or by any route of administration. In some embodiments, the formulation is a sustained release formulation, a controlled release formulation, or a formulation designed for both sustained and controlled release. As used herein, the term “sustained release” refers to release of an active agent such that an approximately constant amount of an active agent becomes available to the subject over time. The phrase “controlled release” is broader, referring to release of an active agent over time that might or might not be at a constant level. Particularly, “controlled release” encompasses situations and formulations where the active ingredient is not necessarily released at a constant rate, but can include increasing release over time, decreasing release over time, and/or constant release with one or more periods of increased release, decreased release, or combinations thereof. Thus, while “sustained release” is a form of “controlled release”, the latter also includes delivery modalities that employ changes in the amount of an active agent that are delivered at different times.

In some embodiments, the sustained release formulation, the controlled release formulation, or the combination thereof is selected from the group consisting of an oral formulation, a peroral formulation, a buccal formulation, an enteral formulation, a pulmonary formulation, a rectal formulation, a vaginal formulation, a nasal formulation, a lingual formulation, a sublingual formulation, an intravenous formulation, an intraarterial formulation, an intracardial formulation, an intramuscular formulation, an intraperitoneal formulation, a transdermal formulation, an intracranial formulation, an intracutaneous formulation, a subcutaneous formulation, an aerosolized formulation, an ocular formulation, an implantable formulation, a depot injection formulation, a transdermal formulation and combinations thereof. In some embodiments, the route of administration is selected from the group consisting of oral, peroral, buccal, enteral, pulmonary, rectal, vaginal, nasal, lingual, sublingual, intravenous, intraarterial, intracardial, intramuscular, intraperitoneal, transdermal, intracranial, intracutaneous, subcutaneous, ocular, via an implant, and via a depot injection. Where applicable, continuous infusion can enhance drug accumulation at a target site (see, e.g., U.S. Pat. No. 6,180,082). See also U.S. Pat. Nos. 3,598,122; 5,016,652; 5,935,975; 6,106,856; 6,162,459; 6,495,605; and 6,582,724; and U.S. Patent Application Publication No. 2006/0188558 for transdermal formulations and methods of delivery of compositions. In some embodiments, the administering is via a route selected from the group consisting of peroral, intravenous, intraperitoneal, inhalation, and intratumoral.

The particular mode of administration of the compositions of the presently disclosed subject matter used in accordance with the methods disclosed herein can depend on various factors, including but not limited to the formulation employed, the severity of the condition to be treated, whether the active agents in the compositions (e.g., an anti-fibrotic) are intended to act locally or systemically, and mechanisms for metabolism or removal of the active agents following administration.

### EXAMPLES

The presently disclosed subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### Materials and Methods for the Examples

Reagents. Recombinant Spike Protein, subunit 1 was purchased from RayBiotech (#230-011101-100, Peachtree Corners, Georgia, United States of America). Xylazine and ketamine were supplied by Henry Schein Animal Health. The BCA Protein assay kit was from Pierce Co. Rabbit total and phosphorylated IKBα (190 9242; #2859) and STAT3 (#4904; #9145) were obtained from Cell Signaling Technology, Inc. IRDye 800CW Goat anti-rabbit and IRDye 680RD Goat anti-mouse were from LI-COR Biosciences. Cytokines levels were measured using Luminex (R&D System). All other reagents were from either Sigma-Aldrich Corporation or from Thermo Fisher Scientific.

Animal model and treatment groups. Animal studies were approved by the Old Dominion University IACUC and adhered to the principles of animal experimentation as published by the American Physiological Society. Male K18-hACE2 transgenic mice (B6·Cg-Tg(K18-ACE2)₂Prlmn/J; Jackson Laboratories; 8-10 weeks old, 24-28 g weight) received either saline (2 μl/g body weight), intratracheally (IT) or S1SP (400 μg/kg in 2 ml/kg body weight, IT), under xylazine (6 mg/kg, IP) and ketamine (60 mg/kg, IP) anesthesia, as described in Marinova et al., 2020. 72 hours after IT administration, all mice were euthanized, subjected to bronchoalveolar lavage fluid, and lung tissue was obtained for further analysis and histology (H&E stain).

### Example 1

### SARS-CoV-2 Spike Protein Subunit 1 (S1SP) Exposure Results in

### Weight Loss and Alveolar Inflammation

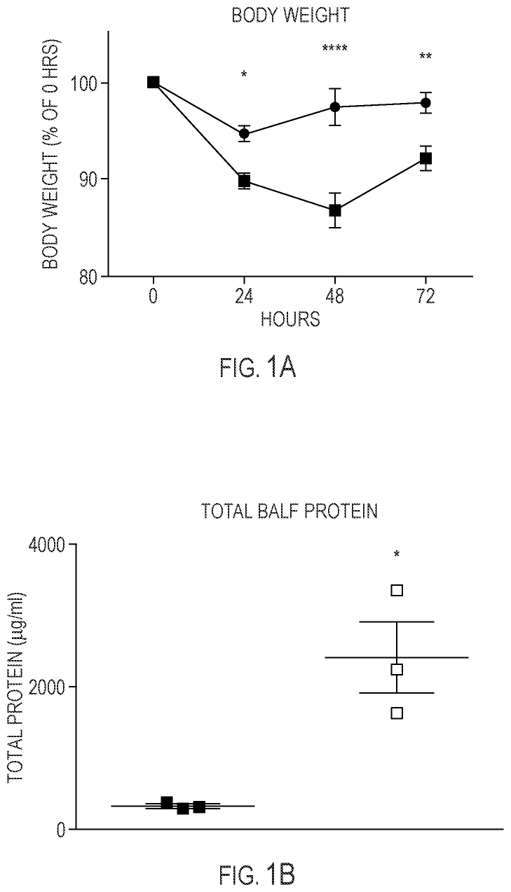

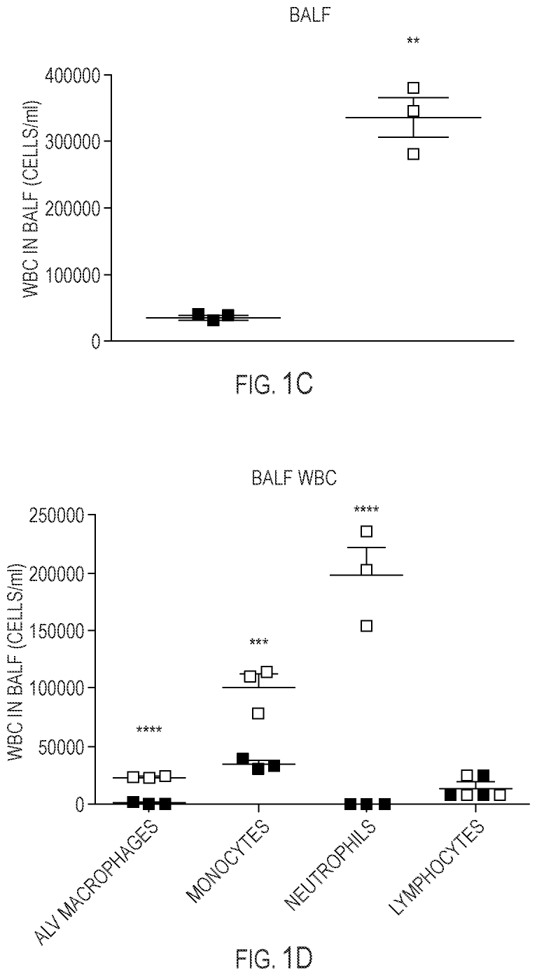

Mice were administered SARS-CoV-2 Spike protein Subunit 1 (S1SP), 400 μg/kg in 2 ml/kg body weight, and weighed at 24, 48, and 72 hours post-instillation. Negative control mice were administered saline. The results are shown in FIG. 1A. Statistically significant reductions in weight were observed at each time point in the mice exposed to S1 SP as compared to negative controls.

At 72 hours post-instillation, mice were euthanized and bronchoalveolar lavage fluid was isolated. Total protein was determined, and as compared to saline controls, S1SP-instilled mice had elevated total BALF protein (see FIG. 1B). Additionally, leukocyte counts were determined in BALF, and as compared to saline controls, S1SP-instilled mice had elevated white blood cell (WBC) counts (see FIG. 1C). A closer analysis of the types of WBCs present in BALF showed that alveolar macrophages, monocytes, and neutrophils were particularly elevated in BALF from S1SP-instilled mice as compared to saline controls, whereas lymphocytes were similar between S1SP-instilled mice and saline controls (see FIG. 1D).

### Example 2

### S1SP Exposure Results in Cytokine Storm

Exposure to the S1 subunit of SARS-CoV-2 Spike Protein induced alveolar “cytokine storm”. Mice were exposed to the S1 subunit of SARS-CoV-2 Spike Protein as set forth herein above, and levels of various cytokines and chemokines were assayed 72 hours after intratracheal instillation of S1SP.

The results are presented in FIG. 2. As compared to saline controls, S1SP-instilled mice experienced statistically significant increases in the levels of IFNγ, IL-1β, IL-6, IL-17, MCP-1, KC, TNFα, and MIP1α.

### Example 3

### S1SP Exposure Results in ALI and Activation of the

### STAT3 and NFκB Inflammatory Pathways

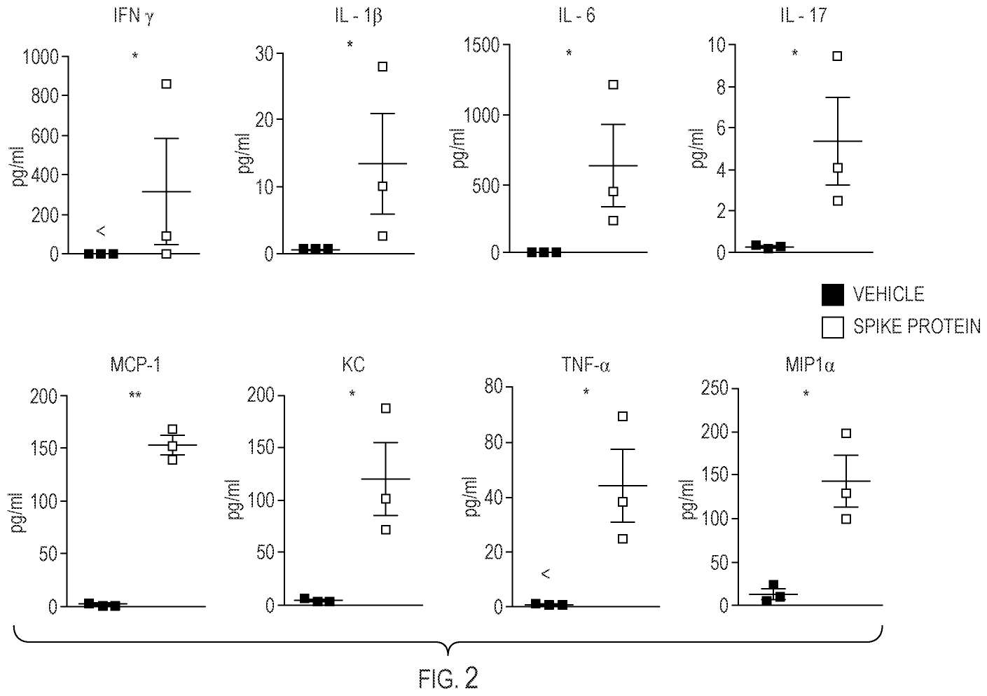

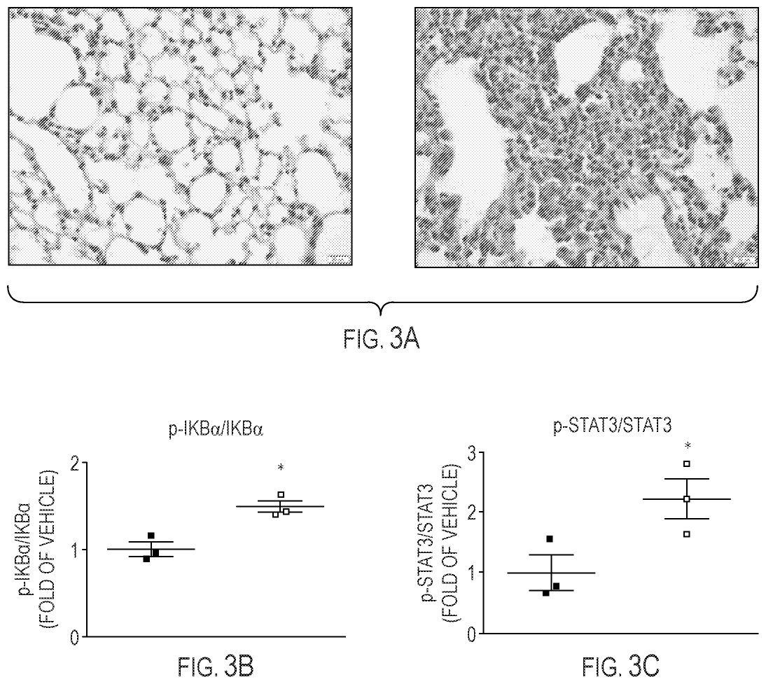

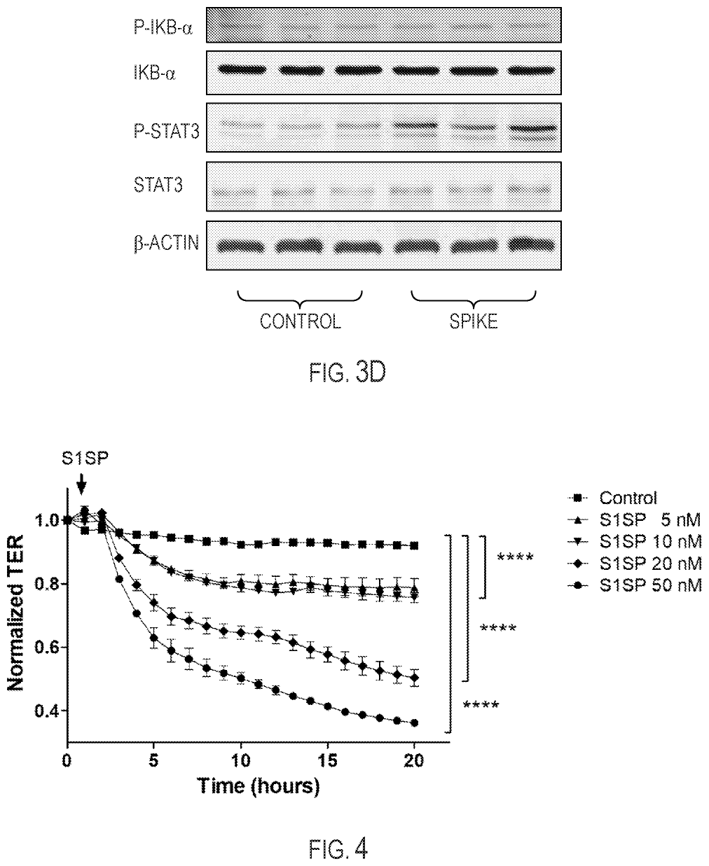

Exposure to the S1 subunit of the SARS-CoV-2 Spike Protein (S1SP) also caused acute lung injury and the activation of the STAT3 and NFκB inflammatory pathways. As shown in FIG. 3A, lung sections of mice treated intratracheally with S1SP (right panel) demonstrated septal thickening, neutrophil infiltration, and edema compared to saline controls (left panel).

Western blot analyses of lung homogenates of mice exposed to S1SP revealed increased phosphorylation of IκBα and STAT3 (see FIGS. 3B-3D), indicating that S1 Sp exposure induced activation of the STAT3 and NFκB inflammatory pathways.

### Example 4

### S1SP Exposure Results in Concentration-dependent Decrease in Barrier Function of Human Lung Microvascular Endothelial Cells

Mice were exposed to S1SP at 10 nM, 20 nM, and 50 nM, or to the full length Spike Protein of SARS-CoV-2. Barrier function was determined by determining normalized transendothelial electrical resistance (TER) at instillation and at various time points up to 40 hours post-instillation. HLMVEC were seeded on gold electrodes in a Electric Cell-substrate Impedance Sensing apparatus (ECIS), model 1600ζθ and let grow till confluency. When a stable resistance was achieved (>800Ω), HLMVEC were exposed to different concentration of SISP. Decreases in resistance indicates increased permeability of the monolayer.

The results are presented in FIG. 4. Exposure to S1SP, particularly at 20 nM and 50 nM, rapidly led to a statistically significant reduction in normalized TER as compared to mice treated with saline.

### Example 5

### Cytokine Profiles in Colon Cancer Cells of Wild Tybe and PTP4A3 Knockout Mice

The cytokine content in colon cancer cells of wildtype PTP4A3 (MsTu WT) and PTP4A3 knockout (MsTu KO) mice was measured by Luminex. PTP4A3 knockout mice had lower levels of TNFα, LIF, MIP-1b, G-CSF, IL-6, and MIP-2 as compared to PTP4A3 wildtype (MsTu WT) mice.

### Example 6

### KVX-053 Blocked Spike Protein (SP)-induced ALI in Mice

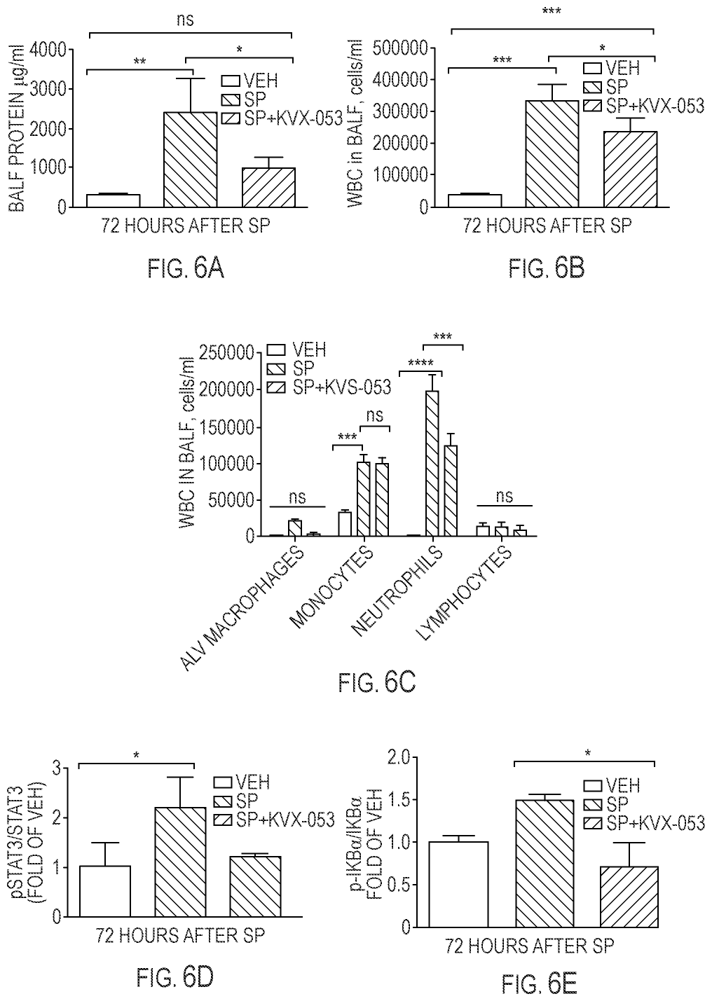

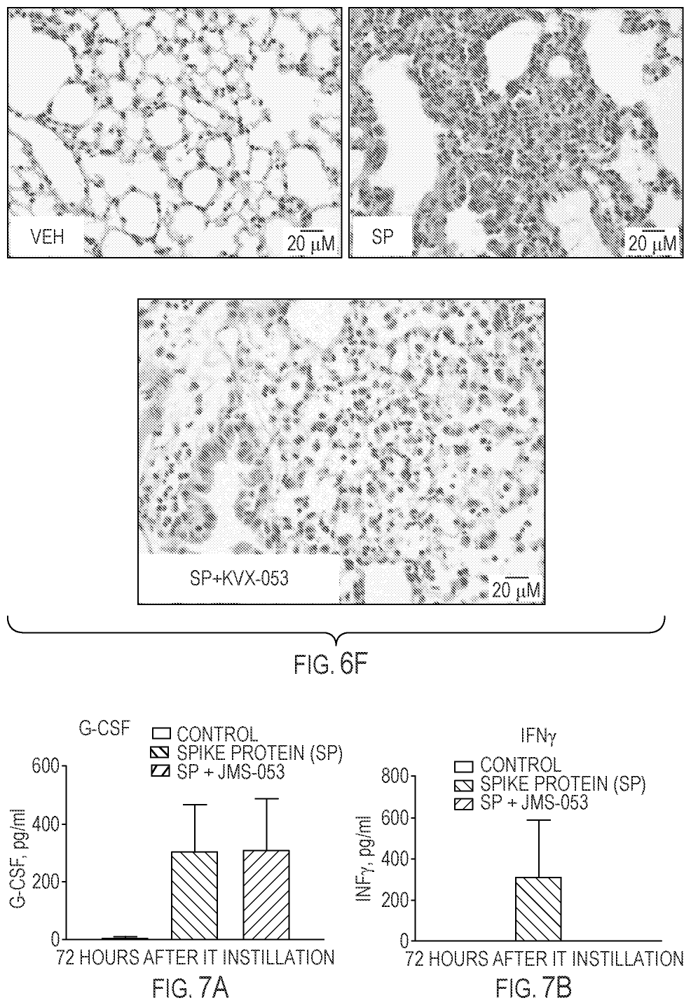

K18-hACE2 mice were treated with either vehicle or KVX-053 after exposure to SP i.t. Total BALF protein, WBC counts in BALF, and markers of inflammation were assayed. The results are presented in FIGS. 6A-6F.

SP increased BALF protein (FIG. 6A), WBC (FIG. 6B), and neutrophil content (FIG. 6C), and activated lung tissue STAT3 (FIG. 6D) and NFκB (FIG. 6E). Furthermore, histological signs of hyaline membrane, alveolar (ALV) septal thickening and hypercellularity (FIG. 6F; H&E stain) were also observed. Post-treatment with KVX-053 completely blocked (FIGS. 6A, 6D, and 6E) or significantly attenuated (FIGS. 6B, 6C, and 6F) signs of SP-induced inflammation and ALI.

### Example 7

### KVX-053 Blocked Spike Protein (S1SP)-induced ALI in Mice

As set forth in more detail herein above, exposure to the S1 subunit of SARS-CoV-2 Spike Protein (S1SP) induced alveolar “cytokine storm”. Whether PTP4A3 inhibition (JMS-053 administration i.p. at 10 mg/kg) could block the S1SP induced alveolar cytokine storm was tested.

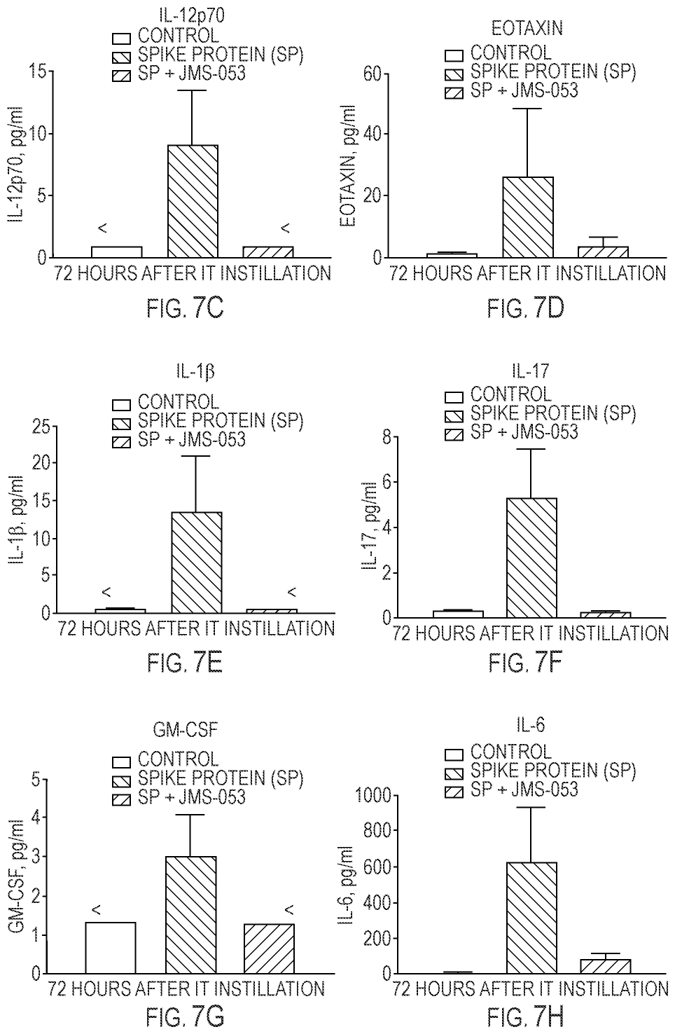

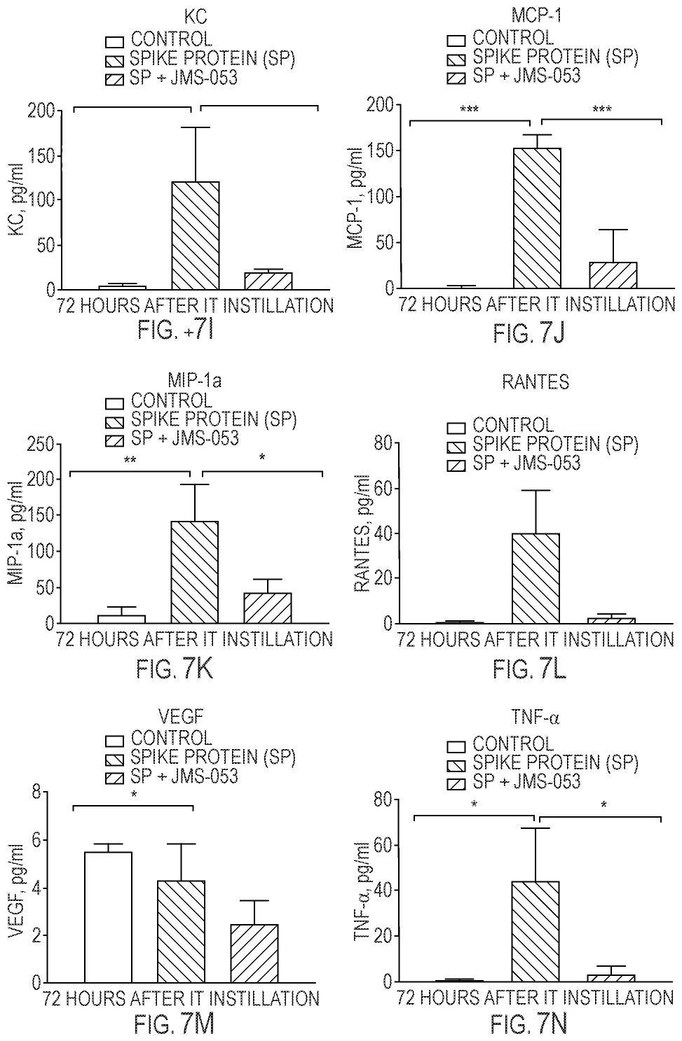

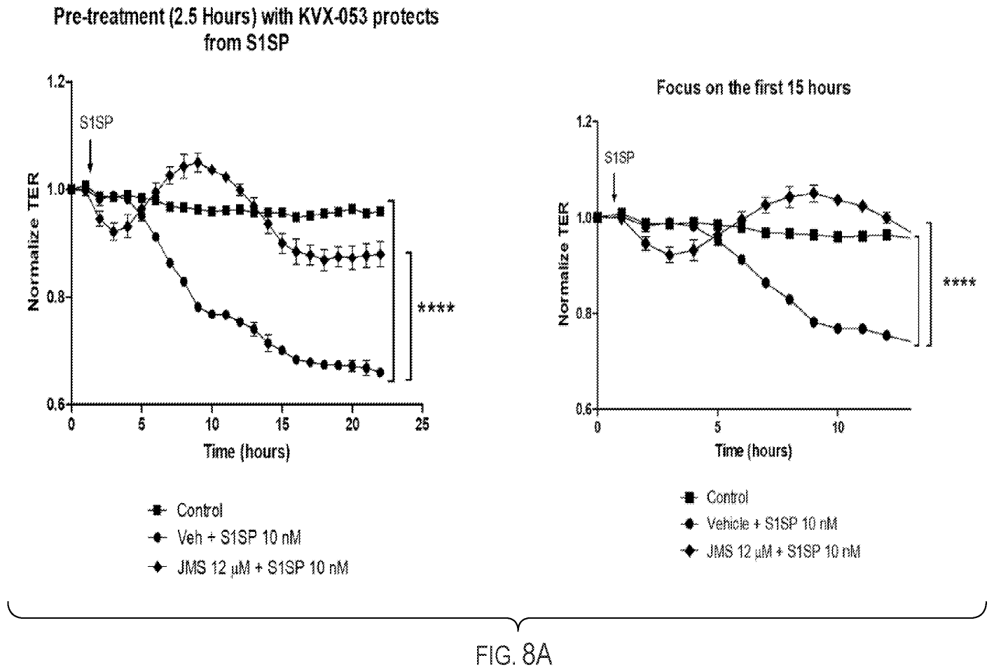

Different pro-inflammatory cytokines in mouse bronchial lavage fluid (BALF) were analyzed 72 hours after intratracheal instillation of S1 SP using a murine 32 plex assay. The results are presented in FIGS. 7A-7N. Pro-inflammatory cytokines that were tested included G-CSF (FIG. 7A), IFNγ (FIG. 7B), I1-12 (FIG. 7C), Eotaxin (FIG. 7D), IL-13 (FIG. 7E), IL-17 (FIG. 7F), GM-CSF (FIG. 7G), IL-6 (FIG. 7H), KC (FIG. 7I), MCP-1 (FIG. 7J), MIP-1a (FIG. 7K), RANTES (FIG. 7L), VEGF (FIG. 7M), and TNF-α (FIG. 7N). As shown in FIGS. 7A-7N, S1SP induced activation of multiple cytokines and chemokines in BALF, and PTP4A3 inhibition by JMS-053 treatment resulted in a decrease in the S1SP-induced increase of each of these cytokines.

### Example 8

### PTP4A3 Inhibition Blocks SP-induced Endothelial Barrier Disruption in Human Lung Endothelial Cells

As described herein above, exposure to SP induced endothelial barrier disruption. The ability of JMS-053 treatment to block SP-induced endothelial barrier disruption was also tested.

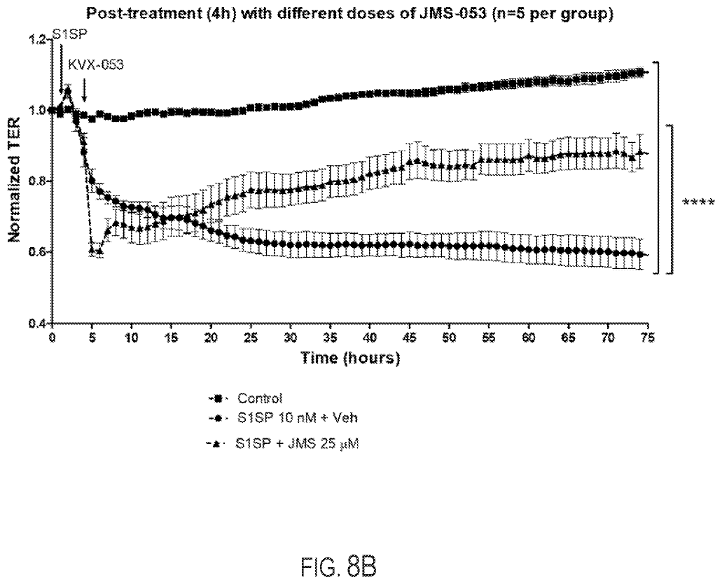

The results are shown in FIGS. 8A and 8B. FIG. 8A shows the results of pre treatment of endothelial cells with KVX-053 for 2.5 or 15 hours prior to exposure to S1SP. FIG. 8B shows the results of pre-treatment of endothelial cells with KVX-053 for 3-4 prior to exposure to S1SP. Summarily, the S1 subunit of the SARS-CoV-2 Spike Protein (S1SP) caused a fast decrease in transendothelial electrical resistance (TER), while KVX-053 caused a concentration- and time-dependent mitigation of the S1SP-induced decrease in TER of human lung microvascular endothelial cells.

### Example 9

### PTP4A3 Inhibition Blocks SP-induced Proinflammatory Chemokine Release In Vivo

The ability of PTP4A3 inhibition to block SP-induced proinflammatory chemokine release was also tested.

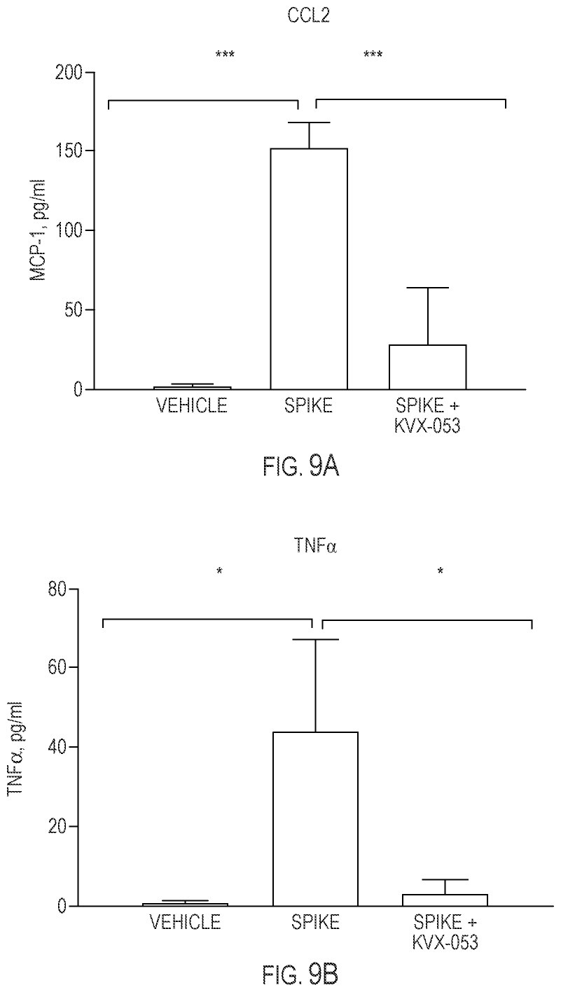

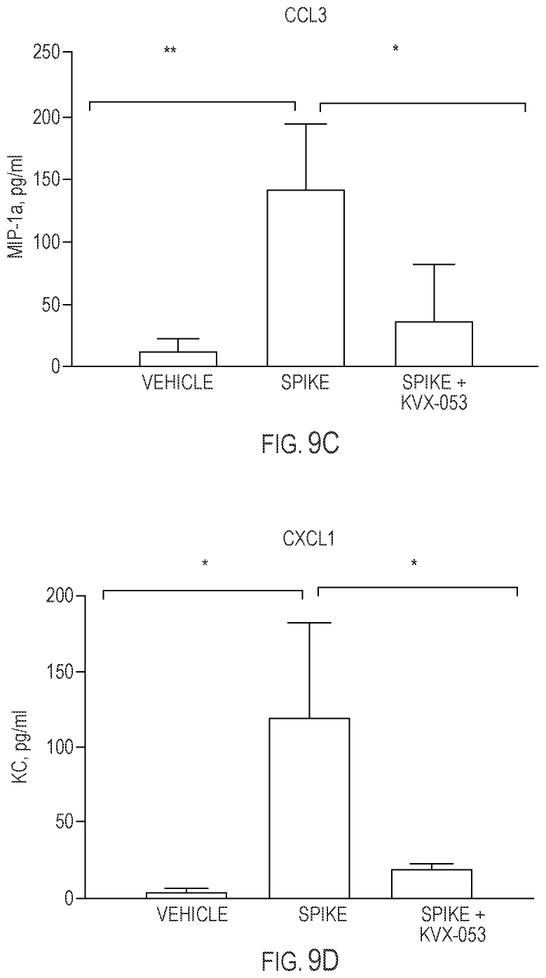

BALF was isolated 72 hours after spike protein instillation and KVX-053 treatment. BALF fluid was then evaluated for cytokine and chemokine levels via Luminex. The results are presented in FIGS. 9A-9D. Shown are four chemokines that were decreased in expression after KVX-053 treatment: MCP-1/CCL2 (FIG. 9A), TNFα (FIG. 9B), MIP1α/CCL3 (FIG. 9C), and KC/CXCL 1 (FIG. 9D).

### Example 10

### PTP4A3 Inhibition Blocks Chemical-induced Lung Injury In Vivo

Lung injury was induced using the sulfur mustard analog 2-chloroethyl ethyl sulfide (CEES) and the effects of KVX-053 were determined.

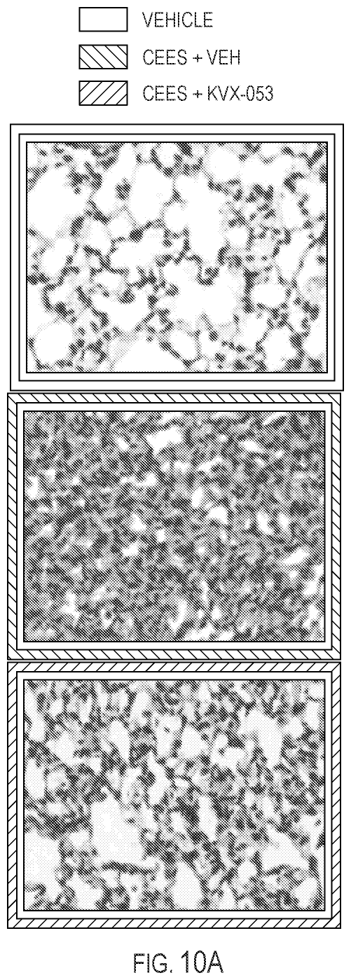

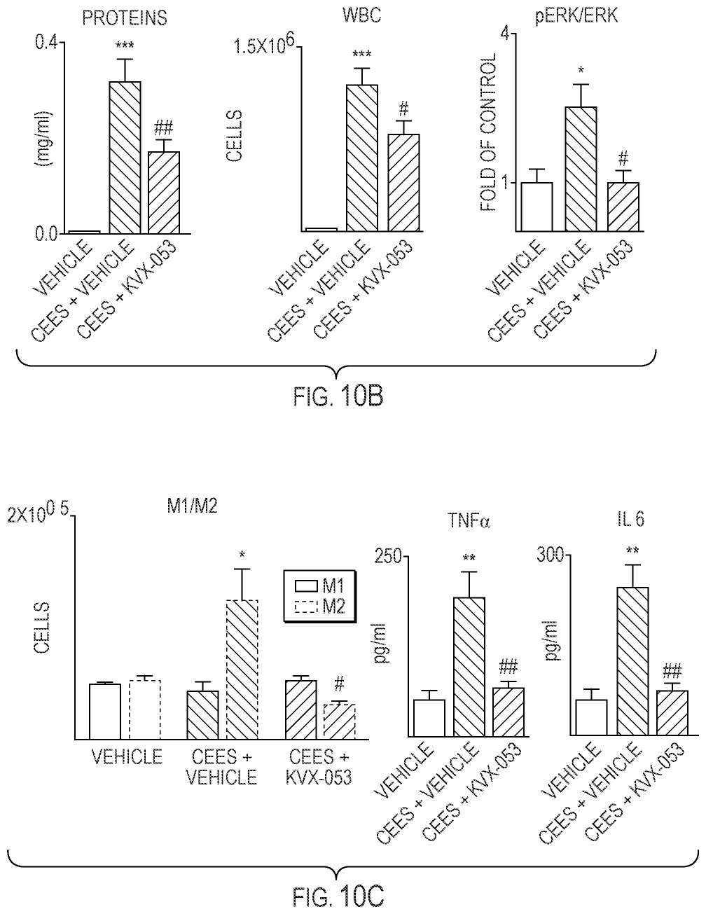

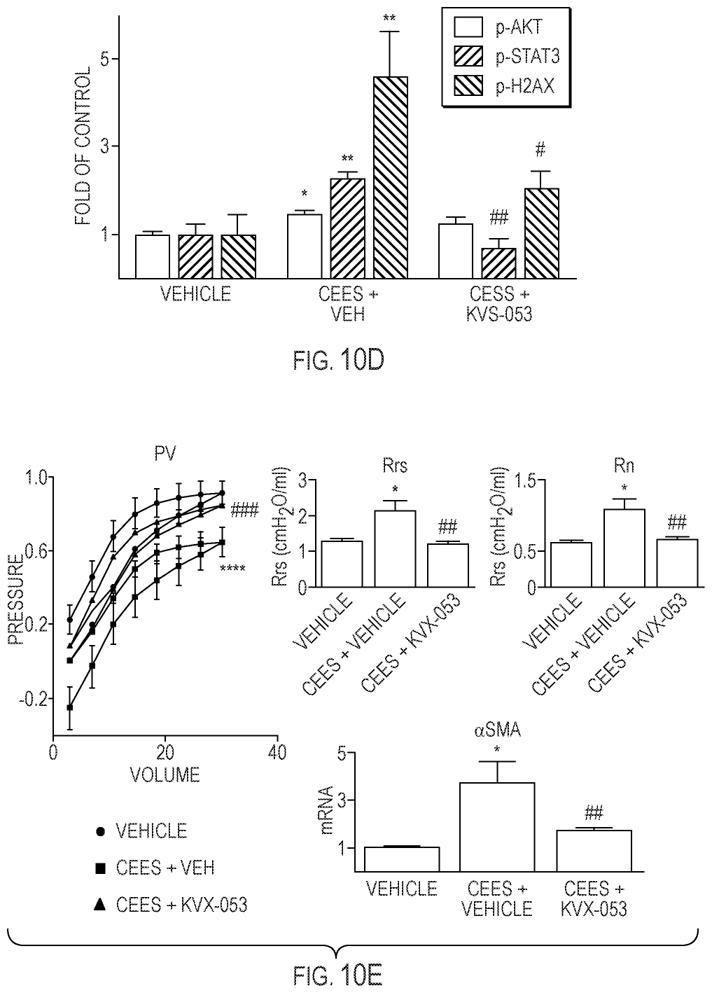

The results are presented in FIGS. 10A-10D. As shown in FIG. 10A, H&E staining shows that CEES induced lung injury, which was reduced by treatment with the PTP4A3 inhibitor KVX-053. FIG. 10B shows that alveolar proteinosis, WBC, and activation of ERK were presented at 6 days post-instillation, the extent of which was also reduced by treatment with KVX-053. CEES elicited persistent increases in TNF-α, IL-6, infiltrative M2 macrophages (CD45⁺, CD11b⁺, CD68⁺; see FIG. 10C). Treatment with KVX-053 reduced these increases (see FIG. 10C).

Furthermore, CEES exposure induced activation of STAT3, AKT, and H2AX as shown by increases in phosphorylated forms of these polypeptides (see FIG. 10D). PTP4A3 inhibition with KVX-053 reduced the activation of these pathways (see FIG. 10D).

Finally, KVX-053 (10 mg/kg/24 h) prevented CEES-induced ALI as shown by a downward shift of Pressure-Volume Loops, increased Systemic and Newtonian resistances (Rrs and Rn), and increased αSMA at 10 days post-instillation (see FIG. 10E).

### Discussion of the Examples 1-10

The strong pathology and lethality of live SARS-CoV-2 virus has been reported in K18-hACE2 mice, which overexpress the human ACE2 gene, but in much milder form in wild type mice (6). This agrees with earlier studies showing that SARS-CoV-1 also requires ACE2 for cellular entry 1 and COVID pathology (Kuba et al., 2005) and with observations that SARS-CoV-1 Spike protein increases levels of IL-8 and MIP-10 in cultured human monocytes-macrophages via NF-κB activation (Dosch et al., 2009). However, to our knowledge, this is the first report of comparable pathogenicity in K18-hACE2 mice by the S1 subunit of the SARS-CoV-2 spike protein. Indeed, our findings of increased alveolocapillary permeability to proteins and inflammatory cells, presence of a “cytokine storm”, activation of two potent inflammatory pathways (STAT3 and NFκB) and histologic evidence of acute lung injury, are in full agreement with recent descriptions of the pathology observed in K18-hACE2 transgenic mice exposed to the live SARS-CoV-2. We consider this important because it provides the means of studying the SARS-CoV-2 associated lung inflammation without the need of employing the highly infective and potentially lethal live virus and therefore without the need of BSL3 conditions, thus making this animal model more widely accessible. Moreover, we have demonstrated that the virally encoded S1 subunit of the SARS-CoV-2 spike protein alone can induce ALI.

### Example 11

SARS-CoV-2 Spike 1 protein increases the permeability of human brain endothelial cells (Buzhdygan et al., 2020) and in this EXAMPLE we have observed similar concentration-dependent changes in human lung microvascular endothelial cells in culture (see FIG. 4). Importantly, the S1 subunit was much more potent than the intact protein which requires cleavage for activity. We previously demonstrated that KVX-053 is a potent, allosteric inhibitor of PTP4A1, 2 and 3 tyrosine phosphatases, which markedly blocks the pulmonary microvascular barrier dysfunction caused by LPS and VEGF (McQueeney et al., 2017). This protective effect is likely due to changes in the actin cytoskeleton, the inhibition of Rac1 and the activation of RhoA and STAT3 (McQueeney et al., 2017; Chong et al., 2019). KVX-053 mitigates VEGF-mediated enhanced permeability when administered 3 h after VEGF exposure. PTP4A3 phosphatase is induced in lung cells 12 h after SARS-CoV infection (Sims et al., 2013) and controls cytokine release (Castro-Sanchez et al., 2018; Aguilar-Sopena et al., 2020). H1N1 and other viruses also induce PTP4A3. IL-6 is one of the most prominent pro-inflammatory cytokines involved in COVID-19 and is known to be involved in a STAT3-PTP4A3 feedforward loop (Chong et al., 2019).

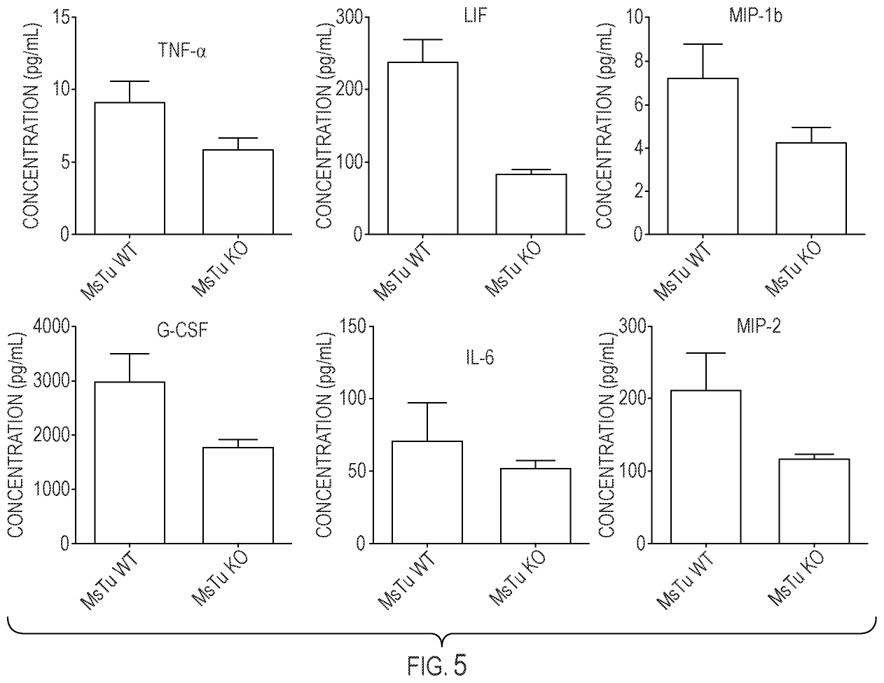

We have also observed that mouse cells in which PTP4A3 has been knocked out (McQueeney et al., 2018) have a reduced level of TNFα, LIF, MIP-1b, MIP-2, IL-6 and G-CSF (see FIG. 5); these are involved in the cytokine storm seen with COVID-19 (Wu & Yang, 2020). The mechanisms responsible for coronavirus-induced inflammation and ALI remain unclear and represent an important therapeutic target, in addition to concurrent efforts for vaccine development and reduction of viral load. The SARS-CoV-2 Spike protein itself exhibits significant pro-inflammatory and pathologic activities that may account, at least in part, for the ALI and inflammation associated with COVID-19 (Liu et al., 2007; Versteeg et al., 2007). SARS-CoV-2 does not effectively infect wildtype laboratory mice due to inefficient interactions between the human Spike protein and the murine ortholog of the human ACE2 (hACE2). Consequently, murine models have and are being developed that enable the use of this valuable species for COVID-19 studies. One of the more promising transgenic models, K18-hACE2, expresses hACE2 under the control of the human keratin 18 promoter. This leads to high hACE2 expression in pulmonary epithelia but also in other cells types (McCray et al., 2007). Intranasal infection of the K18-hACE2 mice with SARS-CoV-2 virus results in severe ALI and ultimately death (Winkler et al., 2020).

In this EXAMPLE, to establish proof of principle, we have challenged male 6-8 weeks old K18-hACE mice with vehicle or 5 μg of the S1 subunit of the human SARS-CoV-2 Spike S protein (SP) administered intratracheally. Saline or 10 mg/kg KVX-053 were then administered at 1, 24 and 48 h later. Mice were euthanized 72 h after SP and examined for signs of inflammation and ALI. As shown in FIGS. 3A-3F, SP caused a dramatic increase in cellular (mostly neutrophil) and protein content of bronchoalveolar lavage fluid (BALF), activation of the NFκB and STAT3 pathways in the whole lung and severe changes in lung architecture, including hyaline membrane, alveolar hypercellularity and septal thickening, all indicative of severe inflammation, alveolocapillary hyperpermeability and ALI. Importantly, KVX-053 post-treatment partly or completely blocked all pathologic evidence of inflammation and ALI.

### Example 12

This EXAMPLE relates to determining the ability of a novel, potent, allosteric, small molecule PTP4A3 inhibitor, KVX-053, to block SARS-CoV-2 Spike 1 protein-mediated loss of pulmonary endothelial barrier function and cytokine release in vitro. The kinetics of disruption of transendothelial membrane barrier function and cytokine release by SARS-CoV-2 Spike 1 protein in the presence or absence of KVX-053 were determine.

The active, 75 kDa S1 subunit of the SARS-CoV-2 Spike protein S is obtained from RayBiotech Life (Peachtree Corners, Georgia, United States of America). Normal human lung microvascular primary endothelial cells are used from a cell bank as described in McQueeney et al., 2017 or, if necessary, from Lifeline Cell Technology to quantify the magnitude and kinetics of changes in endothelial barrier function as measure by differences in the transendothelial electrical resistance (TER) of endothelial monolayers, a method that we have used previously (McQueeney et al., 2017) and that has been repeatedly demonstrated to reflect changes in macromolecular permeability and allows for continuous, quantifiable, real-time monitoring of barrier function (Barabutis et al., 2013). Cells are seeded at 6×10⁴ cells/well on electrode arrays (Applied BioPhysics, Troy, New York, United States of America) and cultured in VascuLife complete medium (Lifeline Cell Technology) to achieve at least an 800Ω baseline steady state resistance and capacitance between 22-29 nanofarads at a frequency of 4000 Hz as described in McQueeney et al., 2017 measured with an ECIS model 1600R detector. We monitor changes in TER for up to 24 h in real-time with at least five concentrations of Spike protein (0.1-50 nM) and use LPS (0.5 Endotoxin Units) as a positive control. TER measured dynamically across the monolayer reflects paracellular permeability, i.e., the combined resistance between the ventral surface of the cell and the electrode, reflective of focal adhesion, as well as resistance between adjacent cells. Intercellular gaps increase current flow and reduce resistance. Thus, changes in TER represent a difference in cell-cell adhesion and/or cell-matrix adhesion. We monitor endothelial cell apoptosis with Annexin V and in situ DNA break extensions, as described in Kondo et al., 1997, while cell death is measured with CellTiter Glo as described in McQueeney et al., 2018. It is notable that other investigators have not observed death of human brain endothelial cells when exposed to 10 nM Spike protein for <72 h (Buzhdygan et al., 2020), nor have we in EXAMPLE 11 (see FIG. 4). A candidate Spike protein concentration is one that yields the largest change in TER without inducing significant apoptosis or cell death. This concentration is used in evaluating the mitigative and preventive effects of KVX-053. Initially we treat human lung microvascular endothelial cells simultaneously with Spike protein and various concentrations of KVX-053 or the inactive control compound KVX-038 (aka JMS-053) (0-5 μM) and monitor TER for 24 h. In subsequent studies we pre-treat cells with KVX-053 or KVX-038 for 0.5-6 h prior to the addition of Spike protein or add KVX-053 or KVX-038 0.5-6 h after S protein exposure, or at the nadir of the TER response to Spike protein.

The SARS-CoV Spike protein directly activates cyclooxygenase-2 (COX-2) expression via both a calcium-dependent pathway, namely PKCα/ERK/NFκB, and a calcium-independent pathway, namely PI3K/PKCα/JNK/CREB, which are linked to inflammation and lung injury (Ackermann et al., 2020). The PTP4A phosphatases regulate actin dynamics most notably during immunological synapse assembly and T cell effector function (Castro-Sanchez et al., 2018). We recently reported that KVX-053 but not KVX-038 blocks cytokine antigen-induced signaling dynamics and cytokine production in Jurkat CD4 T cell (Aguilar-Sopena et al., 2020). Therefore, we examine if the SARS-CoV-2 Spike 1 protein stimulates pro-inflammatory cytokine content and release from human pulmonary microvascular endothelial cells. These studies are important not only for mechanistic reasons but also because they could provide information that could lead to pharmacodynamic endpoint for future clinical trials. The endothelial cells are exposed to concentrations of Spike protein as determined above and cytokines within the endothelial cells and secreted into the medium are determined by Luminex using the Human 41 Plex Panel, that includes IL-6, as this is a major pro-inflammatory cytokine involved in ARDS and is controlled at least partially by PTP4A3 (McQueeney et al., 2017; Jamilloux et al., 2020; Wu & Yang, 2020). In all cases, we confirm changes in protein levels by Western blotting. The cytokine changes detected by the Luminex assay are subjected to higher order network analysis, such as Ingenuity Pathway Analysis and Reactome, as we have done previously (McQueeney et al., 2017; Porterfield et al., 2020) to help define a potential signature of Spike protein and KVX-053 response. We also examine the endothelial cells for STAT3 activation by Western blotting as this is thought to be a key regulator of TH17 responses in cytokine storms of COVID-19 and is regulated by PTP4A3 (Chong et al., 2019; Wu & Yang, 2020). We next treat human lung microvascular endothelial cells simultaneously with Spike protein and various concentrations of KVX-053 or the inactive control compound KVX-038 (0-5 μM) and monitor protein content and release for at least 48 h. Subsequent studies examine cells pre-treated with KVX-053 or KVX-038 for 0.5-6 h prior to the addition of Spike protein or post-treated with KVX-053 or KVX-038 0.5-6 h after Spike protein exposure. Mechanistically, loss of endothelial barrier function is often mediated by activation of RhoA and inhibition of Rac1 (Millar et al., 2015; McQueeney et al., 2017). Therefore, we examine RhoA and Rac1 activation in endothelial cells after Spike protein exposure as a potential biomarker, as described in McQueeney et al., 2017, with or without KVX-053. We also examine ACE2 shedding into the medium by endothelial cells exposed to KVX-053 or KVX-038 using an ACE2 ELISA assay from Novus Biologicals, which has a sensitivity of 78 μg/mL. Collectively, these results provide information about prevention or mitigation by KVX-053 of acute endothelial cell injury from Spike protein engagement.

### Example 13

This EXAMPLE relates to determining the ability of KVX-053 to block SARS-CoV-2 Spike 1 protein-mediated pulmonary alveolar epithelial barrier function and cytokine release in vitro. This Example follows a protocol that is similar to that of Example 12 except we are using normal human alveolar primary epithelial cells rather than endothelial cells. We first determine the kinetics of disruption of transepithelial membrane barrier function and cytokine release by SARS-CoV-2 Spike protein in the presence or absence of KVX-053.

Similar to the microvascular endothelium, the vascular epithelium regulates macromolecular and cellular trafficking through the alveolar walls and enhanced vascular epithelial permeability has been linked to ARDS. We employ recombinant SARS-CoV-2 Wuhan-Hu-1 Spike S1 subunit protein, as described above in Example 12. We use normal human alveolar primary epithelial cells from ScienCell Research Laboratories (Carlsbad, California, United States of America) to quantify the magnitude and kinetics of changes in epithelial barrier function as measure by differences in TER (McQueeney et al., 2017). Cells are cultured in epithelial complete medium and we monitor changes in TER for up to 24 h with at least five concentrations (0.1-50 nM) of Spike protein and use LPS (0.5 Endotoxin Units) as a positive control. We monitor epithelial cell apoptosis and viability as mentioned above (McQueeney et al., 2017; McQueeney et al., 2018). A candidate Spike protein concentration is one that yields the largest change in TER without inducing significant apoptosis or cell death. This concentration is used in evaluating the mitigative and preventive effects of KVX-053. First, we treat epithelial cells simultaneously with Spike protein and various concentrations of KVX-053 or the control compound KVX-038 (0-5 μM). In subsequent studies we pre-treat cells with KVX-053 or KVX-038 for 0.5-6 h prior to the addition of Spike protein or add KVX-053 or KVX-038 0.5-6 h after Spike protein exposure, or at the nadir of the TER response to Spike protein.

We examine if the SARS-CoV-2 Spike protein stimulates pro-inflammatory cytokine content and release from human alveolar epithelial cells. The cells are exposed to concentrations of Spike protein as determined above and the cellular cytokine expression and secretion into the medium are determined by Luminex as mentioned above. As in Example 12, we focus on IL-6 secretion and STAT3 and pSTAT3 levels. In all cases, we confirm changes in protein levels by Western blotting. We will next treat human epithelial cells with various concentrations of KVX-053 or KVX-038 (0-5 μM) simultaneously, before, or after Spike protein addition and monitor protein content and release for at least 48 h including ACE2 shedding. These results provide information about possible prevention or mitigation by KVX-053 of epithelial cell injury from Spike protein engagement.

### Example 14

This EXAMPLE involves determining the ability of KVX-053 to inhibit ALI in mice caused by the SARS-CoV-2 Spike 1 protein. This EXAMPLE tests that the PTP4A3 inhibitor, KVX-053, ameliorates the ALI, inflammation and lung dysfunction induced by SARS-CoV-2 Spike protein in mice. The K18-hACE2 mouse transgenic model, which mimics the human SARS disease (McCray et al., 2007), is used. We obtain 6-8 weeks old male and female B6·Cg-Tg(K18-ACE2)₂Prlmn/J transgenic mice (The Jackson Laboratory). The mice are anesthetized by intraperitoneal injection of ketamine and xylazine. A neck midline incision is then performed to expose the trachea and visualize the correct placement of the catheter. A 20 gauge 1″ catheter is inserted into the trachea. A single injection of Spike protein Subunit 1 (1, 3, 5 or 10 μg in 30 μL saline) is instilled into the trachea and flushed with 100 μL air. Following administration, the catheter is removed, and the surgical opening closed. In initial results, we have demonstrated that 5 μg of Spike protein causes a strong ARDS-like inflammatory response in K18-hACE2 mice (see FIGS. 7A-7N). The PTP4A3 inhibitor, KVX-053 (or the inactive analog, KVX-038, as control) is administered IP at 5 or 10 mg/kg dissolved in 30% modified cyclodextrin (Captisol) with phosphate buffered saline. For pretreatment studies, KVX-053 is given once daily for 2 days prior to Spike protein and for post-treatment, KVX-053 is given once daily for 2 days starting on the day of Spike protein/plasmid administration. Mice are euthanized and analyzed 72 h after Spike protein. The magnitude of the inflammatory response is evaluated in bronchoalveolar lavage fluid (BALF), serum, lung homogenates and histologically, as we have recently reported (Marinova et al., 2019). BALF readouts include total leukocyte and differential counts, total protein concentration, levels of a panel of murine pro-inflammatory cytokines, including IL-6 and TNFα, and ACE2 shedding as measured by ELISA chemiluminescence (Novus Biologicals). The same panel of cytokines are analyzed in serum as will ACE2 shedding. In lung homogenates, we measure NFκB and STAT3 activation, by Western blotting. Myeloperoxidase activity is estimated spectrophotometrically, as index of granulocyte infiltration. In intratracheally fixed and paraffin embedded lungs, thin sections are stained with H&E and myeloperoxidase stains and analyzed by light microscopy to assess lung structural integrity and parenchymal influx of inflammatory cells. Lung function and airway reactivity are evaluated in pentobarbital anesthetized mice, intubated and connected to a Flexivent ventilator (Scireq, Montreal, Canada). A number of parameters are measured (resistance, elastance; static, compliance, and pressure-volume relationships) reflecting lung function, before and after challenge with aerosolized methacholine, to investigate changes in airway reactivity (Chatterjee et al., 2007; Marinova et al., 2019). Initially these experiments are performed in male mice. We then perform the same studies in female K18-hACE2 mice, in an effort to reveal possible gender differences in the effects of either Spike protein or KVX-053.

### Discussion of Examples 11-14

Significant technical difficulties in EXAMPLE 12 are not expected with culturing endothelial cells and measuring TER or generating the recombinant Spike protein based on our previously experience (Barabutis et al., 2013; McQueeney et al., 2017; McQueeney et al., 2018). In the unlikely event that we are unable to observe cytokine release or RhoA/Rac1 signaling after exposure to Spike protein, we employ S1 protein that is labeled with a fluorescent tag, which has previously been shown to bind and enter other cell types to allow us to probe whether pulmonary microvascular endothelial cells differ from other cell types. The fluorescent tag allows us to quantify both cell binding and internalization.

We also use the recently described replication-deficient pseudovirus entry assay (Shang et al., 2020) to track binding and internalization. The described studies with human epithelial cells measuring TER, cytokine and RhoA/Rac1 signaling have pitfalls that are similar to those of endothelial cells and we use similar solutions. In the unlikely event that we are unable to culture or obtain robust TER measurements with human epithelial cells, we can use Calu-3 human lung epithelial cells, which express hACE2 and have been used to study Spike protein entry (Shang et al., 2020).

With respect to EXAMPLE 14, while we know KVX-053 has a long plasma half-life and have selected doses that produces plasma levels of ˜1 μM (McQueeney et al., 2017), the COVID-19 treatment schedule for KVX-053 can be extended with respect to the treatment times or doses beyond what has been presented. That is why the cytokine and other pharmacodynamic endpoints are important and they provide a guide for additional studies. We recognize a number of attractive mouse transgenic models have and are being developed to study SAR-CoV-2 and we monitor their potential use as an alternative to proposed B6·Cg-Tg(K18-ACE2)₂Prlmn/J transgenic mice. An example is the recently described SARS-CoV-2MA mouse in which the mouse ACE2 was reverse genetically engineered to bind to the Spike protein (Dinnon et al., 2020). This transgenic mouse might be useful alternative if an employed transgenic mouse is problematic. Our laboratory has generated and characterized a global PTP4A3 null mice (Zimmerman et al., 2013) and we have also isolated and characterized pulmonary microvascular endothelial cells from both the wildtype and null mice (Zimmerman et al., 2014). These cells can be employed to test the hypothesis that genetic loss of PTP4A3 prevents or mitigates Spike protein-mediated barrier function and cytokine expression and secretion. For pulmonary microvascular epithelial cells, isolation methods are readily available (Lam et al., 2011). The effects of intratracheal Spike protein in the PTP4A3 null mice can be examined to assess the importance of PTP4A3, if funds are available.

The presently disclosed subject matter provides for the development of KVX-053 and related analogs for human therapeutic usage. Upon success of initial experiments, routes of compound injection and formulations that are more likely than the IP route to be adopted in humans are explored, including oral and iv administration. Generated are multigram quantities of research-grade KVX-053 via a recently published continuous flow synthesis methodology (Tasker et al., 2019). Large scale GMP compound production, more complete dose response studies, comprehensive toxicology studies in two species, studies with replication-proficient SARS-CoV-2 are pursued rather than only the Spike protein (requiring BSL-3 facilities, which are available at the University of Virginia and elsewhere). KVX-053 with its novel mechanism of action and broad applicability is highly attractive for use against SARS-CoV-2 if it remains a durable issue or re-emerges or if there are future coronavirus infections or other ARDS-inducing viral infections.

### Example 15

To establish proof of principle, we have continued to examine the cytokine and chemokine composition of the bronchoalveolar lavage fluid (BALF) obtained from K18-hACE mice exposed to vehicle or 5 μg of the S1 subunit of the human SARS-CoV-2 Spike S protein administered intratracheally as described in our proposal (Examples 11 and 14). Saline or 20 mg/kg KVX-053 were then administered at 1 h later. Mice were euthanized 72 h after Spike protein intratracheal instillation. The BALF was isolated and examined by Luminex for chemokine and cytokine composition. As indicated in Table 3 and consistent with the data presented in Examples 11-14 (see FIGS. 6A-6F), the S1 subunit of the human SARS-COV-2 Spike S protein caused a statistically significant increase in six analytes, namely IL-17, KC, MCP-1, MIP-1α, VEGF, and TNF-α. Many of the other pro-inflammatory cytokines and chemokines were also trended toward increases but the number of mice in these preliminary studies (n=3) was small in some cases the basal analyte levels were below the limits of detection (as indicated by <). KVX-053 post-treatment partly or completely blocked five of the six analytes that were significantly altered by the Spike protein and there was a trend toward mitigation with almost all of the other analytes. These encouraging preliminary data suggest that KVX-053 has a profound mitigating effect on the adverse effects of the S1 subunit of the SARS-COV-19 Spike protein and is worthy of further studies with a larger number of mice and different doses and KVX-053 treatment times.

TABLE 3
Cytokine and Chemokine Changes Induced by Intratracheal Instillation
of the Human SARS-COV-2 Spike Protein S1 Subunit (S1SP)
and Mitigation of These Changes with KVX-053

| | | | Spike Protein + |
|---|---|---|---|
| Cytokine/ | Vehicle Control | Spike Protein | KVX-053 |
| Chemokine | pg/mL ± SD (N = 3) | | |
| EOTAXIN | 1.06 ± 0.44 | 26.65 ± 21.93 | 3.98 ± 2.74 |
| GM-CSF | 1.30 ± 0.01 | 3.03 ± 1.89 | 1.30 ± 0.01 |
| IFNg | 0.40 ± 0.11 | 316.6 ± 466.8 | 0.37 ± 0.06 |
| IL-1β | 0.60 ± 0.02< | 13.12 ± 13.02 | 0.59 ± 0.00< |
| IL-6 | 3.67 ± 5.25 | 630.9 ± 521.2 | 85.54 ± 44.95 |
| IL-12p70 | 0.97 ± 0.00< | 9.13 ± 7.60 | 0.97 ± 0.00< |
| IL-17 | 0.27 ± 0.8 | 5.38 ± 3.66* | 0.23 ± 0.02** |
| KC (Keratinocytes- | 3.90 ± 2.12 | 121.5 ± 60.8* | 20.35 ± 1.97** |
| derived chemokine) | | | |
| MCP-1 (CCL3 | 1.18 ± 1.35 | 153.0 ± 14.8* | 30.02 ± 33.91** |
| Antigen) | | | |
| MIP-1α | 12.76 ± 10.06 | 142.8 ± 51.1* | 44.21 ± 17.10 |
| RANTES | 0.70 ± 0.62 | 40.48 ± 31.37 | 2.68 ± 3.00 |
| VEGF | 5.52 ± 2.16 | 0.55 ± 0.17* | 2.54 ± 1.58 |
| TNF-α | 0.79 ± 0.22 | 44.29 ± 22.99* | 3.77 ± 3.10** |

All data were analyzed by ANOVA.
*= p < 0.05 compared to vehicle control.
**= p < −0.05 compared to Spike protein alone.
<= below the limits of detection.

### REFERENCES

All references listed below, as well as all references cited in the instant disclosure, including but not limited to all patents, patent applications and publications thereof, scientific journal articles, and database entries (including all annotations available therein) are incorporated herein by reference in their entireties to the extent that they supplement, explain, provide a background for, or teach methodology, techniques, and/or compositions employed herein.
Ackermann et al. (2020) Pulmonary vascular endothelialitis, thrombosis, and angiogenesis in Covid-19. NEJM 383:120-128.
Aguilar-Sopena et al. (2020) Effect of pharmacological inhibition of the catalytic activity of phosphatases of regenerating liver in early T cell receptor signaling dynamics and IL-2 production. Intl Molec Sci 21:2530.
Altschul et al. (1990a) Basic local alignment search tool. J Mol Biol 215:403-410.
Altschul et al. (1990b) Protein database searches for multiple alignments. Proc Natl Acad Sci USA 87:14:5509-13.
Altschul et al. (1997) Gapped BLAST and PSI-BLAST: a new generation of protein database search programs. Nucleic Acids Res 25:3389-3402.
Bao et al. (2020) The pathogenicity of SARS-CoV-2 in hACE2 transgenic mice. Nature 583(7818):830-833.
Barabutis et al. (2013) LPS induces pp60c-src-mediated tyrosine phosphorylation of Hsp90 in lung vascular endothelial cells and mouse lung. Am J Physiol Lung Cell Mol Physiol 304:L883-893.
Barabutis et al. (2018) Wild-type p53 enhances endothelial barrier function by mediating RACI signalling and RhoA inhibition. J Cell Mol Med 22:1792-1804.
Berkow et al. (1997) The Merck Manual of Medical Information, Home ed., Merck Research Laboratories, Whitehouse Station, New Jersey, United States of America.
Bersini et al. (2020) Transcriptional and functional changes of the human microvasculature during physiological aging and Alzheimer Disease. Adv Biosyst 4:e2000044.
Bird et al. (1988) Single-chain antigen-binding proteins. Science 242:423-426
Buzhdygan et al. (2020) The SARS-CoV-2 spike protein alters barrier function in 2D static and 3D microfluidic in vitro models of the human blood-brain barrier Neurobiol Dis 146:105131.
Castro-Sanchez et al. (2018) Phosphatase of regenerating liver-1 (PRL-1) regulates actin dynamics during immunological synapse assembly and T cell effector function. Front Immunol 9:2655.
Catravas et al. (1981) Biochemical markers of bleomycin toxicity: clearance of 14C-5-hydroxytryptamine and 3H-norepinephrine of rabbit lung in vivo. J Pharmacol Exptl Therap 217:524-529.
Catravas et al. (1983) Pulmonary endothelial dysfunction in the presence or absence of interstitial injury induced by intratracheally-injected bleomycin in rabbits. Amer Rev Resp Dis 128:740-746.
Catravas et al. (2010) Harvesting, identification and barrier function of human lung microvascular endothelial cells. Vascul Pharmacol 52:175-181.
Chatterjee et al. (2007) Heat shock protein 90 inhibitors prolong survival, attenuate inflammation, and reduce lung injury in murine sepsis. Amer J Resp Critical Care Med 176:667-675.
Chen et al. (2020) Clinical and immunological features of severe and moderate coronavirus disease 2019. J Clin Invest 130:2620-2629.
Chong et al. (2019) IL6 Promotes a STAT3-PRL3 Feedforward Loop via SHP2 Repression in Multiple Myeloma. Cancer Res 79:4679-4688.
Colunga Biancatelli et al. (2021) The SARS-CoV-2 spike protein subunit S1 induces COVID-19-like acute lung injury in K18-hACE2 transgenic mice and barrier dysfunction in human endothelial cells. Am J Physiol Lung Cell Mol Physiol 321:L477-L484.
Devereux et al. (1984) A comprehensive set of sequence analysis programs for the VAX. Nucl Acids Res 12:387.
Dinnon et al. (2020) A mouse-adapted SARS-CoV-2 model for the evaluation of COVID-19 medical countermeasures. Nature 586:560-566.
Donoghue et al. (2000) A novel angiotensin-converting enzyme-related carboxypeptidase (ACE2) converts angiotensin I to angiotensin 1-9. Circ Res 87:E1-9.
Dosch et al. (2009) SARS coronavirus spike protein-induced innate immune response occurs via activation of the NF-kappaB pathway in human monocyte macrophages in vitro. Virus Research 142(1-2):19-27.
Duch et al. (1998) Volatile anesthetics significantly suppress central and peripheral mammalian sodium channels. Toxicol Lett 100-101:255-263.
Ebadi (1998) CRC Desk Reference of Clinical Pharmacology. CRC Press, Boca Raton, Florida, United States of America.
Freireich et al., (1966) Quantitative comparison of toxicity of anticancer agents in mouse, rat, hamster, dog, monkey, and man. Cancer Chemother Rep, 50:219-244.
Genaro (ed.) (1985) Remington's Pharmaceutical Sciences, Mack Publishing Co., Easton, Pennsylvania, United States of America.
Gennaro (ed.) (1990) Remington's Pharmaceutical Sciences, 18th ed., Mack Pub. Co., Easton, Pennsylvania, United States of America,
Gennaro (ed.) (2003) Remington: The Science and Practice of Pharmacy, 20th edition Lippincott, Williams & Wilkins, Philadelphia, Pennsylvania, United States of America.
Gerbino (2005) Remington: The Science and Practice of Pharmacy. 21st Edition. Lippincott Williams & Wilkins, Philadelphia, Pennsylvania, United States of America.
Goodman et al. (1996) Goodman & Gilman's the Pharmacological Basis of Therapeutics, 9th ed., McGraw-Hill Health Professions Division, New York, New York, United States of America.
Gross & Mienhofer (eds.) (1981) The Peptides, Vol. 3, Academic Press, New York, New York, United States of America, pages 3-88.
Haga et al. (2008) Modulation of TNF-alphaconverting enzyme by the spike protein of SARS-CoV and ACE2 induces TNF-alpha production and facilitates viral entry. Proc Natl Acad Sci USA 105:7809-7814.
Harlow & Lane, 1988, Antibodies, A Laboratory Manual, Cold Spring Harbor Publications, Cold Spring Harbor, New York, United States of America.
Hoffman et al. (2020) SARS-CoV-2 Cell Entry Depends on ACE2 and TMPRSS2 and Is Blocked by a Clinically Proven Protease Inhibitor. Cell 181:271-280.
Huston et al. (1988) Protein engineering of antibody binding sites: recovery of specific activity in an anti-digoxin single-chain Fv analogue produced in Escherichia coli. Proc Natl Acad Sci USA 85:5879.
Jamilloux et al. (2020) Should we stimulate or suppress immune responses in COVID-19? Cytokine and anti-cytokine interventions. Autoimmun Rev 17(7):102567.
Jones et al (1986) Replacing the complementarity-determining regions in a human antibody with those from a mouse. Nature 321:522.
Karlin & Altschul (1990) Methods for assessing the statistical significance of molecular sequence features by using general scoring schemes. Proc Natl Acad Sci USA 87(6):2264-2268.
Karlin & Altschul (1993) Applications and statistics for multiple high-scoring segments in molecular sequences. Proc Natl Acad Sci USA 90(12):5873-5877.
Katzung (2001) Basic & Clinical Pharmacology, 8th ed., Lange Medical Books/McGraw-Hill Medical Pub. Division, New York, New York, United States of America.
Kondo et al. (1997) Enhanced apoptosis in metallothionein null cells. Molec Pharmacol 52:195-201.
Kuba et al. (2005) A crucial role of angiotensin converting enzyme 2 (ACE2) in SARS coronavirus-induced lung injury. Nature Medicinell(8):875-879.
Lam et al. (2011) Isolation of mouse respiratory epithelial cells and exposure to cigarette smoke at air liquid interface. J Vis Exp 48:2513.
Liu et al. (2007) Spike protein of SARS-CoV stimulates cyclooxygenase-2 expression via both calcium-dependent and calcium-independent protein kinase C pathways. FASEB J 21:1586-1596.
Marinova et al. (2019) Acute exposure of mice to hydrochloric acid leads to the development of chronic lung injury and pulmonary fibrosis. Inhal Toxicol 31:147-160.
Marinova et al. (2020) Post-treatment with a heat shock protein 90 inhibitor prevents chronic lung injury and pulmonary fibrosis, following acute exposure of mice to HCl. Experimental Lung Research 46(6):203-216.
McCray et al. (2007) Lethal infection of K18-hACE2 mice infected with severe acute respiratory syndrome coronavirus. J Virol 81:813-821.
McQueeney et al. (2017) Targeting ovarian cancer and endothelium with an allosteric PTP4A3 phosphatase inhibitor. Oncotarget 9:8223-8240.
McQueeney et al. (2018) A chemical genetics approach identifies PTP4A3 as a regulator of colon cancer cell adhesion. FASEB J 32:5661-5673.
Michaud et al. (2020) ACE2 as a Therapeutic Target for COVID-19; its Role in Infectious Processes and Regulation by Modulators of the RAAS System. Journal of Clinical Medicine 9(7):2096.
Millar et al. (2015) The pulmonary endothelium in acute respiratory distress syndrome: insights and therapeutic opportunities. Thorax 71:462-473.
Munster et al. (2020) Respiratory disease in rhesus macaques inoculated with SARS-CoV-2. Nature 585(7824):268-272.
Oladunni et al. (2020) Lethality of SARS-CoV-2 infection in K18 human angiotensin-converting enzyme 2 transgenic mice. Nature Communications 11(1):6122.
PCT International Publication No. WO 93/25521.
Porterfield et al. (2020) A three-dimensional dementia model reveals spontaneous cell cycle re-entry and a senescence-associated secretory phenotype. Neurobiol Aging 90:125-134.
Remington et al. (1975) Remington's Pharmaceutical Sciences, 15th ed., Mack Pub. Co., Easton, Pennsylvania, United States of America.
Riechmann et al. (1988) Reshaping human antibodies for therapy. Nature 332(6162):323-327.
Rivellese & Prediletto (2020) ACE2 at the centre of COVID-19 from paucisymptomatic infections to severe pneumonia. Autoimmun Rev 19(6):102536.
Shang et al. (2020) Cell entry mechanisms of SARS-CoV-2. Proc Nat Acad Sci USA 117:11727-11734.
Shi et al. (2020) COVID-19 infection: the perspectives on immune responses. Cell Death Differ 27:1451-1454.
Sia et al. (2020) Pathogenesis and transmission of SARS-CoV-2 in golden hamsters. Nature 583(7818):834-838.
Sims et al. (2013) Release of severe acute respiratory syndrome coronavirus nuclear import block enhances host transcription in human lung cells. J Virol 87:3885-3902.
Speight & Holford (eds.) (1997) Avery's Drug Treatment: A Guide to the Properties, Choice, Therapeutic Use and Economic Value of Drugs in Disease Management, 4th ed., Adis International, Philadelphia, Pennsylvania, United States of America.
Sun et al. (2020) Cytokine storm intervention in the early stages of COVID-19 pneumonia. Cytokine Growth Factor Rev 53:38-42.
Tasker et al. (2019) In-flow photooxygenation of aminothienopyridinones generates iminopyridinedione PTP4A3 phosphatase inhibitors. Org Biomol Chem 17:2448-2466.
U.S. Patent Application Publication Nos. 2003/0017534, 2006/0188558, 2018/0298087, 2018/0312588, 2018/0346564, 2019/0151448, 2022/0017534.
U.S. Pat. Nos. 3,598,122; 4,816,567; 5,016,652; 5,234,933; 5,326,902; 5,482,856; 5,935,975; 6,106,856; 6,162,459; 6,180,082; 6,479,284; 6,495,605; 6,582,724; 6,677,436; 7,060,808; 7,906,625; 8,398,980; 8,436,150; 8,796,439; 10,253,111; 10,308,663.
Versteeg et al. (2007) The coronavirus spike protein induces endoplasmic reticulum stress and upregulation of intracellular chemokine mRNA concentrations. J Virol 81:10981-10990.
Winkler et al. (2020) SARS-CoV-2 infection of human ACE2-transgenic mice causes severe lung inflammation and impaired function. Nature Immunology 21(11):1327-1335.
Winter & Milstein (1991) Man-made antibodies. Nature 349(6307):293-299.
Wu & Yang (2020) TH17 responses in cytokine storm of COVID-19: An emerging target of JAK2 inhibitor Fedratinib. J Microbiol Immunol Infect 53(3):368-370.
Zhou et al. (2020) A pneumonia outbreak associated with a new coronavirus of probable bat origin. Nature 579(7798):270-273.
Zimmerman et al. (2013) Targeted deletion of the metastasis-associated phophatase Ptp4a3 (PRL-3) suppresses murine colon cancer. PloS one 8:e58300.
Zimmerman et al. (2014) PTP4A3 phosphatase promotes VEGF signaling and enables endothelial cell motility. J Biol Chem 289:5904-5913.

While the presently disclosed subject matter has been disclosed with reference to specific embodiments, it is apparent that other embodiments and variations of the presently disclosed subject matter may be devised by others skilled in the art without departing from the true spirit and scope of the presently disclosed subject matter.

1. A method for treating and/or preventing a disease, disorder, and/or condition associated with viral infection in a subject, the method comprising, consisting essentially of, or consisting of administering to the subject an effective amount of a protein tyrosine phosphatase 4A3 (PTP4A3) inhibitor.

2. The method of claim 1, wherein the disease, disorder, and/or condition comprises lung damage.

3. The method of claim 1, wherein the disease,
disorder, and/or condition associated with viral infection is selected from the group consisting of acute lung injury (ALI), acute respiratory distress syndrome (ARDS).

4. The method of claim 1, wherein the PTP4A3 inhibitor is selected from the group consisting of 7-imino-2-phenylthieno[3,2-c]pyridine-4,6(5H,7H)-dione, thienopyridone, and compounds comprising the following general structure: 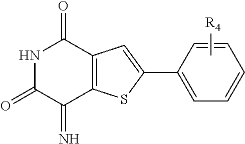
wherein:
R⁴ is selected from the group consisting of H, —OH, —OC₁₋₄ alkyl, —OR^{a}, trifluoroC₁₋₄alkoxy, —SC₁₋₄ alkyl, —SR^{a}, —SO₂—C₁-_alkyl, —SO₂R^{a}, —SOC₁₋₄ alkyl, —SOR^{a}, —SO₂NHR^{b}, —NR^{C}R^{d}, halo, —C₁₋₁₂ alkyl, —C₂₋₆ alkenyl, —C₂₋₆ alkynyl, —C₃₋₆cycloalkyl, phenyl, benzyl, monocyclic heteroaryl optionally substituted with R^{b}, —C₃₋₆ cycloalkyl, —C₄₋₇ heterocycloalkyl containing one or two of O, S, and N, —OC(O)R^{b}, —OC(O)R^{b}, —P(O)(OR^{b})₁₋₂, —P(S)OR^{b})₁₋₂, —P(O)(NR^{C}R^{d})₁₋₂, —P(S)(NR^{C}R^{d})₁₋₂, —O(CH₂—CH₂—O)₁₋₄CH₃, —CN, —NO₂, —C(O)C₁₋₄alkyl, and —C(O)—R^{b},
R^{a} is selected from the group consisting of-C₆cycloalkyl, —C₂alkenyl, —C₂₋₆ alkynyl, phenyl, benzyl, and monocyclic heteroaryl, wherein the phenyl, benzyl, or monocyclic heteroaryl is optionally substituted with R^{b};
R^{b} in each instance is independently selected from the group consisting of H, halo, —OH, —COOH, —C₁₋₁₂alkyl, phenyl, benzyl, and monocyclic heteroaryl, wherein the phenyl, benzyl, or monocylic heteroaryl is optionally substituted with —C₂₋₆alkenyl, —C₂₋₆alkynyl, trifluroC₁₋₄ alkoxy, —OC₁₋₄alkyl, —O(CH₂—CH₂—O)₁₋₄CH₃, —O-phenyl, —O—benzyl, —NC₁₋₄alkyl, —N-phenyl, and —N-benzyl, or —N-monocyclic heteroaryl,
R^{C} and R^{d} are each independently selected from the group consisting of H, —C₁₋₄ alkyl, —C(O)—C₁₋₄alkyl, —C(O)—R^{e}, —C₁₋₄ alkyl-R^{e}, —SO₂—R^{a}, —SO₂—C₁₋₄ alkyl, phenyl, benzyl, and monocyclic heteroaryl, wherein the phenyl, benzyl or monocyclic heteroaryl is optionally substituted with R^{b}, or
or R^{C} and R^{d} taken together with the nitrogen to which they are attached represent an optionally substituted monocyclic heterocycloalkyl containing one or more of O, S, and N,
pharmaceutically acceptable salts thereof, prodrugs thereof, metabolites thereof,
or any combination thereof.

5. The method of claim 1, wherein the viral infection is a SARS-CoV-2 infection and/or an influenza A infection, optionally an infection with an H1N1 subtype of influenza A.

6. A method for reducing or inhibiting virus-induced alveolar inflammation and/or damage, the method comprising administering to a subject in need thereof an effective amount of a protein tyrosine phosphatase 4A3 (PTP4A3) inhibitor.

7. The method of claim 6, wherein the disease, disorder, and/or condition is induced by infection with SARS-CoV-2.

8. The method of claim 6, wherein the PTP4A3 inhibitor is selected from the group consisting of 7-imino-2-phenylthieno[3,2-c]pyridine-4,6(5H,7H)-dione, thienopyridone, and compounds comprising the following general structure: 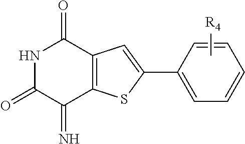
wherein:
R⁴ is selected from the group consisting of H, —OH, —OC₁₋₄ alkyl, —OR^{a}, trifluoroC₁₋₄alkoxy, —SC₁₋₄ alkyl, —SR^{a}, —SO₂—C₁-_alkyl, —SO₂R^{a}, —SOC₁₋₄ alkyl, —SOR^{a}, —SO₂NHR^{b}, —NR^{C}R^{d}, halo, —C₁₋₁₂ alkyl, —C₂₋₆ alkenyl, —C₂₋₆ alkynyl, —C₃₋₆cycloalkyl, phenyl, benzyl, monocyclic heteroaryl optionally substituted with R^{b}, —C₃₋₆ cycloalkyl, —C₄₋₇ heterocycloalkyl containing one or two of O, S, and N, —OC(O)R^{b}, —OC(O)R^{b}, —P(O)(OR^{b})₁₋₂, —P(S)OR^{b})₁₋₂, —P(O)(NR^{C}R^{d})₁₋₂, —P(S)(NR^{C}R^{d})₁₋₂, —O(CH₂—CH₂—O)₁₋₄CH₃, —CN, —NO₂, —C(O)C₁₋₄alkyl, and —C(O)—R^{b},
R^{a} is selected from the group consisting of-C₆cycloalkyl, —C₂alkenyl, —C₂₋₆ alkynyl, phenyl, benzyl, and monocyclic heteroaryl, wherein the phenyl, benzyl, or monocyclic heteroaryl is optionally substituted with R^{b};
R^{b} in each instance is independently selected from the group consisting of H, halo, —OH, —COOH, —C₁₋₁₂alkyl, phenyl, benzyl, and monocyclic heteroaryl, wherein the phenyl, benzyl, or monocylic heteroaryl is optionally substituted with —C₂₋₆alkenyl, —C₂₋₆alkynyl, trifluroC₁₋₄ alkoxy, —OC₁₋₄alkyl, —O(CH₂—CH₂—O)₁₋₄CH₃, —O-phenyl, —O—benzyl, —NC₁₋₄alkyl, —N-phenyl, and —N-benzyl, or —N-monocyclic heteroaryl,
R^{C} and R^{d} are each independently selected from the group consisting of H, —C₁₋₄ alkyl, —C(O)—C₁₋₄alkyl, —C(O)—R^{e}, —C₁₋₄ alkyl-R^{e}, —SO₂—R^{a}, —SO₂—C₁₋₄ alkyl, phenyl, benzyl, and monocyclic heteroaryl, wherein the phenyl, benzyl or monocyclic heteroaryl is optionally substituted with R^{b}, or
or R^{C} and R^{d} taken together with the nitrogen to which they are attached represent an optionally substituted monocyclic heterocycloalkyl containing one or more of O, S, and N.
pharmaceutically acceptable salts thereof, prodrugs thereof, metabolites thereof, or any combination thereof.

9. A method for reducing or inhibiting induction of an inflammatory cytokine and/or chemokine by a viral infection in a subject, optionally a SARS-CoV-2 infection, the method comprising administering to a subject in need thereof an effective amount of a protein tyrosine phosphatase 4A3 (PTP4A3) inhibitor.

10. The method of claim 9, wherein the inflammatory cytokine and/or chemokine is selected from the group consisting of IFNγ, IL-1p, IL-6, IL-17, MCP-1, KC, TNFα, MIP-1a, CLL2, CCL3, and CXCL1.

11. The method of claim 9, wherein the PTP4A3 inhibitor is selected from the group consisting of 7-imino-2-phenylthieno[3,2-c]pyridine-4,6(5H,7H)-dione, thienopyridone, and compounds comprising the following general structure: 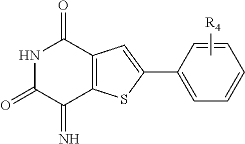
wherein:
R⁴ is selected from the group consisting of H, —OH, —OC₁₋₄ alkyl, —OR^{a}, trifluoroC₁₋₄alkoxy, —SC₁₋₄ alkyl, —SR^{a}, —SO₂—C₁-_alkyl, —SO₂R^{a}, —SOC₁₋₄ alkyl, —SOR^{a}, —SO₂NHR^{b}, —NR^{C}R^{d}, halo, —C₁₋₁₂ alkyl, —C₂₋₆ alkenyl, —C₂₋₆ alkynyl, —C₃₋₆cycloalkyl, phenyl, benzyl, monocyclic heteroaryl optionally substituted with R^{b}, —C₃₋₆ cycloalkyl, —C₄₋₇ heterocycloalkyl containing one or two of O, S, and N, —OC(O)R^{b}, —OC(O)R^{b}, —P(O)(OR^{b})₁₋₂, —P(S)OR^{b})₁₋₂, —P(O)(NR^{C}R^{d})₁₋₂, —P(S)(NR^{C}R^{d})₁₋₂, —O(CH₂—CH₂—O)₁₋₄CH₃, —CN, —NO₂, —C(O)C₁₋₄alkyl, and —C(O)—R^{b},
R^{a} is selected from the group consisting of-C₆cycloalkyl, —C₂alkenyl, —C₂₋₆ alkynyl, phenyl, benzyl, and monocyclic heteroaryl, wherein the phenyl, benzyl, or monocyclic heteroaryl is optionally substituted with R^{b};
R^{b} in each instance is independently selected from the group consisting of H, halo, —OH, —COOH, —C₁₋₁₂alkyl, phenyl, benzyl, and monocyclic heteroaryl, wherein the phenyl, benzyl, or monocyclic heteroaryl is optionally substituted with —C₂₋₆alkenyl, —C₂₋₆alkynyl, trifluroC₁₋₄ alkoxy, —OC₁₋₄alkyl, —O(CH₂—CH₂—O)₁₋₄CH₃, —O-phenyl, —O—benzyl, —NC₁₋₄alkyl, —N-phenyl, and —N-benzyl, or —N-monocyclic heteroaryl,
R^{C} and R^{d} are each independently selected from the group consisting of H, —C₁₋₄ alkyl, —C(O)—C₁₋₄alkyl, —C(O)—R^{e}, —C₁₋₄ alkyl-R^{e}, —SO₂—R^{a}, —SO₂—C₁₋₄ alkyl, phenyl, benzyl, and monocyclic heteroaryl, wherein the phenyl, benzyl or monocyclic heteroaryl is optionally substituted with R^{b}, or
or R^{C} and R^{d} taken together with the nitrogen to which they are attached represent an optionally substituted monocyclic heterocycloalkyl containing one or more of O, S, and N,
pharmaceutically acceptable salts thereof, prodrugs thereof, metabolites thereof, or any combination thereof.

12. A method for reducing or inhibiting a pulmonary disease, disorder, and/or condition associated with a viral infection in a subject, or pulmonary damage resulting therefrom, the method comprising administering to a subject in need thereof an effective amount of a protein tyrosine phosphatase 4A3 (PTP4A3) inhibitor.

13. The method of claim 12, wherein the pulmonary disease, disorder, and/or condition is alveolar thickening, neutrophil infiltration, endothelial barrier disruption, fibrosis, or any combination thereof.

14. The method of claim 12, wherein the PTP4A3 inhibitor is selected from the group consisting of 7-imino-2-phenylthieno[3,2-c]pyridine-4,6(5H,7H)-dione, thienopyridone, and compounds comprising the following general structure: 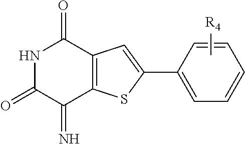
wherein:
R⁴ is selected from the group consisting of H, —OH, —OC₁₋₄ alkyl, —OR^{a}, trifluoroC₁₋₄alkoxy, —SC₁₋₄ alkyl, —SR^{a}, —SO₂—C₁₋₄ alkyl, —SO₂R^{a}, —SOC₁₋₄alkyl, —SOR^{a}, —SO₂NHR_{b}, —NR^{C}R^{d}, halo, —C₁₋₁₂alkyl, —C₂₋₆alkenyl, —C₂₋₆alkynyl, —C₃-6cycloalkyl, phenyl, benzyl, monocyclic heteroaryl optionally substituted with R^{b}, —C₃₋₆ cycloalkyl, —C₄₋₂heterocycloalkyl containing one or two of O, S, and N, —OC(O)R^{b}, —OC(O)R^{b}, —P(O)(OR^{b})₁-2, —P(S)(OR^{b})₁₋₂, —P(O)(NR^{C}R^{d})₁₋₂, —P(S)(NR^{C}R^{d})₁₋₂, —O(CH₂—CH₂—O)₁₋₄CH₃, —CN, —NO₂, —C(O)C₁₋₄alkyl, and —C(O)—R^{b},
R^{a} is selected from the group consisting of —C₃₋₆cycloalkyl, —C₂₋₆ alkenyl, —C₂-6 alkynyl, phenyl, benzyl, and monocyclic heteroaryl, wherein the phenyl, benzyl, or monocyclic heteroaryl is optionally substituted with R^{b};
R^{b} in each instance is independently selected from the group consisting of H, halo, —OH, —COOH, —C₁₋₁₂alkyl, phenyl, benzyl, and monocyclic heteroaryl, wherein the phenyl, benzyl, or monocyclic heteroaryl is optionally substituted with —C₂₋₆alkenyl, —C₂₋₆ alkynyl, trifluroC₁₋₄ alkoxy, —OC₁₋₄ alkyl, —O(CH₂—CH₂—O)₁₋₄ CH₃, —O-phenyl, —O— benzyl, —NC₁₋₄ alkyl, —N-phenyl, and —N-benzyl, or —N-monocyclic heteroaryl;
R^{C} and R^{d} are each independently selected from the group consisting of H, —C₁₋₄ alkyl, —C(O)—C₁₋₄alkyl, —C(O)—R^{e}, —C₁₋₄ alkyl-R^{a}, —SO₂—R^{a}, —SO₂—C₁₋₄ alkyl, phenyl, benzyl, and monocyclic heteroaryl, wherein the phenyl, benzyl, or monocyclic heteroaryl is optionally substituted with R^{b}, or
or R^{C} and R^{d} taken together with the nitrogen to which they are attached represent an optionally substituted monocyclic heterocycloalkyl containing one or more of O, S, and N.
pharmaceutically acceptable salts thereof, prodrugs thereof, metabolites thereof, or any combination thereof.

15. The method of claim 12, wherein the viral infection is a SARS-CoV-2 infection and/or an influenza A infection, optionally an infection with an H1N1 subtype of influenza A.

16. The method of claim 1, wherein the administering is oral, intravenous, intramuscular, subcutaneous, intraperitoneal, intranasal, pulmonary, or any combination thereof.

17. (canceled)

18. (canceled)

19. (canceled)

20. A composition comprising a protein tyrosine phosphatase 4A3 (PTP4A3) inhibitor for use in a method for treating and/or preventing a disease, disorder, and/or condition associated with viral infection in a subject, optionally a SARS-CoV-2 infection in a subject, the method comprising administering to a subject infected with or at risk for infection with a virus via a route and in an amount effective for treating and/or preventing the disease, disorder, and/or condition associated with the viral infection in the subject.

21. The composition for use of claim 20, wherein the PTP4A3 inhibitor is selected from the group consisting of 7-imino-2-phenylthieno[3,2-c]pyridine-4,6(5H,7H)-dione, thienopyridone, and compounds comprising the following general structure: 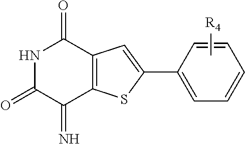
wherein:
R⁴ is selected from the group consisting of H, —OH, —OC₁₋₄ alkyl, —OR^{a}, trifluoroC₁₋₄alkoxy, —SC₁₋₄ alkyl, —SR^{a}, —SO₂—C₁₋₄ alkyl, —SO₂R^{a}, —SOC₁₋₄alkyl, —SOR^{a}, —SO₂NHR_{b}, —NR^{C}R^{d}, halo, —C₁₋₁₂alkyl, —C₂₋₆alkenyl, —C₂₋₆alkynyl, —C₃-₆cycloalkyl, phenyl, benzyl, monocyclic heteroaryl optionally substituted with R^{b}, —C₃₋₆ cycloalkyl, —C₄₋₂heterocycloalkyl containing one or two of O, S, and N, —OC(O)R^{b}, —OC(O)R^{b}, —P(O)(OR^{b})₁-2, —P(S)(OR^{b})₁₋₂, —P(O)(NR^{C}R^{d})₁₋₂, —P(S)(NR^{C}R^{d})₁₋₂, —O(CH₂—CH₂—O)₁₋₄CH₃, —CN, —NO₂, —C(O)C₁₋₄alkyl, and —C(O)—R^{b},
R^{a} is selected from the group consisting of —C₃₋₆cycloalkyl, —C₂₋₆ alkenyl, —C₂-6 alkynyl, phenyl, benzyl, and monocyclic heteroaryl, wherein the phenyl, benzyl, or monocyclic heteroaryl is optionally substituted with R^{b};
R^{b} in each instance is independently selected from the group consisting of H, halo, —OH, —COOH, —C₁₋₁₂alkyl, phenyl, benzyl, and monocyclic heteroaryl, wherein the phenyl, benzyl, or monocyclic heteroaryl is optionally substituted with —C₂₋₆alkenyl, —C₂₋₆ alkynyl, trifluroC₁₋₄ alkoxy, —OC₁₋₄ alkyl, —O(CH₂—CH₂—O)₁₋₄ CH₃, —O-phenyl, —O— benzyl, —NC₁₋₄ alkyl, —N-phenyl, and —N-benzyl, or —N-monocyclic heteroaryl;
R^{C} and R^{d} are each independently selected from the group consisting of H, —C₁₋₄ alkyl, —C(O)—C₁₋₄alkyl, —C(O)—R^{e}, —C₁₋₄ alkyl-R^{a}, —SO₂—R^{a}, —SO₂—C₁₋₄ alkyl, phenyl, benzyl, and monocyclic heteroaryl, wherein the phenyl, benzyl, or monocyclic heteroaryl is optionally substituted with R^{b}, or
or R^{C} and R^{d} taken together with the nitrogen to which they are attached represent an optionally substituted monocyclic heterocycloalkyl containing one or more of O, S, and N,
pharmaceutically acceptable salts thereof, prodrugs thereof, metabolites thereof, or any combination thereof.

22. The composition for use of claim 20, wherein the disease, disorder, and/or condition associated with the viral infection comprises alveolar inflammation, alveolar thickening, neutrophil infiltration into the lung, endothelial barrier disruption, fibrosis, or any combination thereof.

23. A method for preventing and/or treating chemical damage to a lung in a subject, the method comprising administering to a subject in need thereof an effective amount of a protein tyrosine phosphatase 4A3 (PTP4A3) inhibitor.

24. The method of claim 23, wherein the chemical damage results in alveolar thickening, neutrophil infiltration, endothelial barrier disruption, fibrosis, or any combination thereof.

25. The method of claim 23, wherein the PTP4A3 inhibitor is selected from the group consisting of 7-imino-2-phenylthieno[3,2-c]pyridine-4,6(5H,7H)-dione, thienopyridone, and compounds comprising the following general structure: 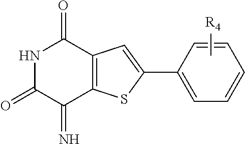
R⁴ is selected from the group consisting of H, —OH, —OC₁₋₄ alkyl, —OR^{a}, trifluoroC₁₋₄alkoxy, —SC₁₋₄ alkyl, —SR^{a}, —SO₂—C₁₋₄ alkyl, —SO₂R^{a}, —SOC₁₋₄alkyl, —SOR^{a}, —SO₂NHR_{b}, —NR^{C}R^{d}, halo, —C₁₋₁₂alkyl, —C₂₋₆alkenyl, —C₂₋₆alkynyl, —C₃-6cycloalkyl, phenyl, benzyl, monocyclic heteroaryl optionally substituted with R^{b}, —C₃₋₆ cycloalkyl, —C₄₋₂heterocycloalkyl containing one or two of O, S, and N, —OC(O)R^{b}, —OC(O)R^{b}, —P(O)(OR^{b})₁-2, —P(S)(OR^{b})₁₋₂, —P(O)(NR^{C}R^{d})₁₋₂, —P(S)(NR^{C}R^{d})₁₋₂, —O(CH₂—CH₂—O)₁₋₄CH₃, —CN, —NO₂, —C(O)C₁₋₄alkyl, and —C(O)—R^{b},
R^{a} is selected from the group consisting of —C₃₋₆cycloalkyl, —C₂₋₆ alkenyl, —C₂-6 alkynyl, phenyl, benzyl, and monocyclic heteroaryl, wherein the phenyl, benzyl, or monocyclic heteroaryl is optionally substituted with R^{b};
R^{b} in each instance is independently selected from the group consisting of H, halo, —OH, —COOH, —C₁₋₁₂alkyl, phenyl, benzyl, and monocyclic heteroaryl, wherein the phenyl, benzyl, or monocyclic heteroaryl is optionally substituted with —C₂₋₆alkenyl, —C₂₋₆ alkynyl, trifluroC₁₋₄ alkoxy, —OC₁₋₄ alkyl, —O(CH₂—CH₂—O)₁₋₄ CH₃, —O-phenyl, —O— benzyl, —NC₁₋₄ alkyl, —N-phenyl, and —N-benzyl, or —N-monocyclic heteroaryl;
R^{C} and R^{d} are each independently selected from the group consisting of H, —C₁₋₄ alkyl, —C(O)—C₁₋₄alkyl, —C(O)—R^{e}, —C₁₋₄ alkyl-R^{a}, —SO₂—R^{a}, —SO₂—C₁₋₄ alkyl, phenyl, benzyl, and monocyclic heteroaryl, wherein the phenyl, benzyl, or monocyclic heteroaryl is optionally substituted with R^{b}, or
or R^{C} and R^{d} taken together with the nitrogen to which they are attached represent an optionally substituted monocyclic heterocycloalkyl containing one or more of O, S, and N,
pharmaceutically acceptable salts thereof, prodrugs thereof, metabolites thereof, or any combination thereof.

26. The method of claim 23, wherein the administering is oral, intravenous, intramuscular, subcutaneous, intraperitoneal, intranasal, pulmonary, or any combination thereof.


SEQUENCE LISTING (4 sequences)

SEQ ID NO: 1 - DNA, 2849 bp, Homo sapiens
  CDS (918)..(1439)
gtgcgcgagggcgcgcgcgtcccgagccctccacccgtcgtgccggcgccgcccggaccgccagatgctgtgtgctgtggacccacctggggttcatggagtgggccacggggcccagccctaagcactgctgcgcccagggtcgccgcgcctcctgctgaggggtccccgtgccactggctctcaccattgccctcgcctgccgatggcctctgctgcccagcctggggccagctctaccgcctgagccccctgccccactccaggactcaccgtaccccgatggggtaacgtgacacaggccccacacgtcagaggccgctgtccccacggccactgcccgtgacccctggcccaaggcagctggagttggttcagttcaagttcattcttcctctggcccttgggggcttggggcccacctctgagtgaagggggctgtctgcccatccaccaatgtggagagggcgcccccggtgtggggtccagctctggacactgcttggcggccgggttcactttgagtttttaagttttctttgctgagcttttttggttgttctttttattttttgcctctttatgactatccagctctgagagacgggagtttggagttgcccgctttactttggttgggttggggggggcggcgggctgttttgttccttttcttttttaagagttgggttttcttttttaattatccaaacagtgggcagcttcctcccccacacccaagtatttgcacaatatttgtgcggggtatgggggtgggtttttaaatctcgtttctcttggacaagcacagggatctcgttctcctcattttttgggggtgtgtggggacttctcaggtcgtgtccccagccttctctgcagtcccttctgccctgccgggcccgtcgggaggcgccatggctcggatgaaccgcccggccccggtggaggtgagctacaaacacatgcgcttcctcatcacccacaaccccaccaacgccacgctcagcaccttcattgaggacctgaagaagtacggggctaccactgtggtgcgtgtgtgtgaagtgacctatgacaaaacgccgctggagaaggatggcatcaccgttgtggactggccgtttgacgatggggcgcccccgcccggcaaggtagtggaagactggctgagcctggtgaaggccaagttctgtgaggcccccggcagctgcgtggctgtgcactgcgtggcgggcctgggccgggctccagtccttgtggcgctggcccttattgagagcgggatgaagtacgaggacgccatccagttcatccgccagaagcgccgcggagccatcaacagcaagcagctcacctacctggagaaataccggcccaaacagaggctgcggttcaaagacccacacacgcacaagacccggtgctgcgttatgtagctcaggaccttggctgggcctggtcgtcatgtaggtcaggaccttggctggacctggaggccctgcccagccctgctctgcccagcccagcaggggctccaggccttggctggccccacatcgccttttcctccccgacacctccgtgcacttgtgtccgaggagcgaggagcccctcgggccctgggtggcctctgggccctttctcctgtctccgccactccctctggcggcgctggccgtggctctgtctctctgaggtgggtcgggcgccctctgcccgccccctcccacaccagccaggctggtctcctctagcctgtttgttgtggggtgggggtatattttgtaaccactgggcccccagcccctcttttgcgaccccttgtcctgacctgttctcggcaccttaaattattagaccccggggcagtcaggtgctccggacacccgaaggcaataaaacaggagccgtggccgtgtgtgtggagtgggctacagcgtcaggcggggcgggctggtggcctgggggccccagaggctgctgtctggatcctgggctggtgcccaggatggggctcccgcgtgctcttgcgctgccctctggtggccgctctgggtccttgcaccccgacccaggggccagcctgccctgtcctgtcctgataccgaggtgggagccctgccttggccagggtggccgtgttgacggttcttgggactgtgacattggaaggcgaggcaggtcaccagcactgtcctctgcaggatgggctgggattcatttggcagcttctcagggcctgtgtccggctggttggtccctgtgctgcccaaaccaggtgtccacatttccggctccgaggcgcagagaagggggcaggtggtggcttgggtggaggaagtcaccatccatcagcccagggagggagggtgccacctgggcacctggggctggatgtgagaggcctggaccagggcccgccggagggcgtggaccagatgctcatgtgttcctgggtgcagtgtctgtgttgggggctggccccaccctgggccggggtgcatggagggcatggccccagcggggaggaaggtgggcctagggctggctccagggtgtggagagcctgggagtggtctccgtcctggggccccaggaggttcccgcaaggagcgactggggcaggtgctggaggaggtcagtggacaagatggggagatgtggaaaccccaaaagccccttctcaggcagccctgcccccaagaccgacagatggctaagggggccgcagacctggctcccccagctcctgtgtggagaaagggcaacagctgtcccggatggttattctctcctttcctcaaacacatttggaactcaagtaaatccaatgcatgttgggtgaa

SEQ ID NO: 2 - protein, 173 aa, Homo sapiens
MARMNRPAPVEVSYKHMRFLITHNPTNATLSTFIEDLKKYGATTVVRVCEVTYDKTPLEKDGITVVDWPFDDGAPPPGKVVEDWLSLVKAKFCEAPGSCVAVHCVAGLGRAPVLVALALIESGMKYEDAIQFIRQKRRGAINSKQLTYLEKYRPKQRLRFKDPHTHKTRCCVM

SEQ ID NO: 3 - DNA, 2710 bp, Homo sapiens
  CDS (854)..(1300)
atgctgtgtgctgtggacccacctggggttcatggagtgggccacggggcccagccctaagcactgctgcgcccagggtcgccgcgcctcctgctgaggggtccccgtgccactggctctcaccattgccctcgcctgccgatggcctctgctgcccagcctggggccagctctaccgcctgagccccctgccccactccaggactcaccgtaccccgatggggtaacgtgacacaggccccacacgtcagaggccgctgtccccacggccactgcccgtgacccctggcccaaggcagctggagttggttcagttcaagttcattcttcctctggcccttgggggcttggggcccacctctgagtgaagggggctgtctgcccatccaccaatgtggagagggcgcccccggtgtggggtccagctctggacactgcttggcggccgggttcactttgagtttttaagttttctttgctgagcttttttggttgttctttttattttttgcctctttatgactatccagctctgagagacgggagtttggagttgcccgctttactttggttgggttggggggggcggcgggctgttttgttccttttcttttttaagagttgggttttcttttttaattatccaaacagtgggcagcttcctcccccacacccaagtatttgcacaatatttgtgcggggtatgggggtgggtttttaaatctcgtttctcttggacaagcacagggatctcgttctcctcattttttgggggtgtgtggggacttctcaggtcgtgtccccagccttctctgcagtcccttctgccctgccgggcccgtcgggaggcgccatggctcggatgaaccgcccggccccggtggaggtgagctacaaacacatgcgcttcctcatcacccacaaccccaccaacgccacgctcagcaccttcattgaggacctgaagaagtacggggctaccactgtggtgcgtgtgtgtgaagtgacctatgacaaaacgccgctggagaaggatggcatcaccgttgtggactggccgtttgacgatggggcgcccccgcccggcaaggtagtggaagactggctgagcctggtgaaggccaagttctgtgaggcccccggcagctgcgtggctgtgcactgcgtggcgggcctgggccggaagcgccgcggagccatcaacagcaagcagctcacctacctggagaaataccggcccaaacagaggctgcggttcaaagacccacacacgcacaagacccggtgctgcgttatgtagctcaggaccttggctgggcctggtcgtcatgtaggtcaggaccttggctggacctggaggccctgcccagccctgctctgcccagcccagcaggggctccaggccttggctggccccacatcgccttttcctccccgacacctccgtgcacttgtgtccgaggagcgaggagcccctcgggccctgggtggcctctgggccctttctcctgtctccgccactccctctggcggcgctggccgtggctctgtctctctgaggtgggtcgggcgccctctgcccgccccctcccacaccagccaggctggtctcctctagcctgtttgttgtggggtgggggtatattttgtaaccactgggcccccagcccctcttttgcgaccccttgtcctgacctgttctcggcaccttaaattattagaccccggggcagtcaggtgctccggacacccgaaggcaataaaacaggagccgtggccgtgtgtgtggagtgggctacagcgtcaggcggggcgggctggtggcctgggggccccagaggctgctgtctggatcctgggctggtgcccaggatggggctcccgcgtgctcttgcgctgccctctggtggccgctctgggtccttgcaccccgacccaggggccagcctgccctgtcctgtcctgataccgaggtgggagccctgccttggccagggtggccgtgttgacggttcttgggactgtgacattggaaggcgaggcaggtcaccagcactgtcctctgcaggatgggctgggattcatttggcagcttctcagggcctgtgtccggctggttggtccctgtgctgcccaaaccaggtgtccacatttccggctccgaggcgcagagaagggggcaggtggtggcttgggtggaggaagtcaccatccatcagcccagggagggagggtgccacctgggcacctggggctggatgtgagaggcctggaccagggcccgccggagggcgtggaccagatgctcatgtgttcctgggtgcagtgtctgtgttgggggctggccccaccctgggccggggtgcatggagggcatggccccagcggggaggaaggtgggcctagggctggctccagggtgtggagagcctgggagtggtctccgtcctggggccccaggaggttcccgcaaggagcgactggggcaggtgctggaggaggtcagtggacaagatggggagatgtggaaaccccaaaagccccttctcaggcagccctgcccccaagaccgacagatggctaagggggccgcagacctggctcccccagctcctgtgtggagaaagggcaacagctgtcccggatggttattctctcctttcctcaaacacatttggaactcaagtaaatccaatgcatgttgggtgaa

SEQ ID NO: 4 - protein, 148 aa, Homo sapiens
MARMNRPAPVEVSYKHMRFLITHNPTNATLSTFIEDLKKYGATTVVRVCEVTYDKTPLEKDGITVVDWPFDDGAPPPGKVVEDWLSLVKAKFCEAPGSCVAVHCVAGLGRKRRGAINSKQLTYLEKYRPKQRLRFKDPHTHKTRCCVM